(19) United States
(12) Patent Application Publication
(10) Pub. No.: US 2010/0136552 A1
(43) Pub. Date: Jun. 3, 2010

(54) Methods and compositions for DMXL-associated mental retardation

(75) Inventors: Madhuri Hegde, Lilburn, GA (US); Margaret Adam, Seattle, WA (US)

Correspondence Address: MYERS BIGEL SIBLEY & SAJOVEC, PO BOX 37428, RALEIGH, NC, 27627, US

(73) Assignee: Emory University

(21) Appl. No.: 12/589,355

(22) Filed: Oct. 22, 2009

Related U.S. Application Data
(60) Provisional application No. 61/197,168, filed on Oct. 24, 2008.

(51) Int. Cl. C12Q 1/68; C12P 19/34

(52) U.S. Cl. 435/6; 435/91.2

(57) ABSTRACT

The present invention provides a method of identifying a human subject as having an increased likelihood of having DMXL-associated mental retardation, comprising detecting, in a nucleic acid sample from the subject, a mutation in a nucleotide sequence encoding DMXL1 and/or a mutation in a nucleotide sequence encoding DMXL2. The present invention further provides a method of identifying a human subject as having an increased likelihood of having DMXL-associated mental retardation, comprising detecting, in a sample from the subject, a mutation in a DMXL1 protein and/or a mutation in a DMXL2 protein.

### STATEMENT OF PRIORITY

This application claims the benefit, under 35 U.S.C. §119(e), of U.S. Provisional Application Ser. No. 61/197,168, filed Oct. 24, 2008, the entire contents of which are incorporated by reference herein.

### INCORPORATION OF SEQUENCE LISTING ON COMPACT DISK

The entire contents of the compact disk filed in identical duplicate herewith and containing one file entitled “9799-2 FINAL Sequence Listing CD copy” (604 kb; created Oct. 22, 2009) are incorporated by reference herein.

### FIELD OF THE INVENTION

The present invention provides methods and compositions directed to detection of mutations in the DMXL1 and/or DMXL2 gene and identification of subjects having an increased likelihood of having DMXL-associated mental retardation and/or of having DMXL-associated mental retardation.

### BACKGROUND OF THE INVENTION

Mental retardation is characterized both by a significantly below-average score on a test of mental ability or intelligence and by limitations in the ability to function in areas of daily life, such as communication, self-care, and getting along in social situations and school activities. Mental retardation is sometimes referred to as a cognitive or intellectual disability [1-6].

Mental retardation can start anytime before a child reaches the age of 18 years. It may result from injury, disease, or a brain abnormality, which can occur before a child is born or during childhood. For many children, the cause of their mental retardation is not known [7, 8]. Some of the most common known causes of mental retardation are Down syndrome, fetal alcohol syndrome, and fragile X syndrome, all of which are determined before birth [9-11]. Other causes that take place before a child is born include genetic conditions (such as Cri-du-chat syndrome or Prader-Willi syndrome), infections (such as congenital cytomegalovirus), or birth defects that affect the brain (such as hydrocephalus or cortical atrophy) [12, 13]. Other causes of mental retardation, such as asphyxia, happen while a baby is being born or soon after birth. Still other causes do not occur until a child is older.

There are several hundred disorders associated with mental retardation, and many of them play a causal role in mental retardation. The American Association on Mental Retardation subdivides the disorders that may be associated with mental retardation into three general areas: prenatal, perinatal, and postnatal causes [14]. It should also be noted that mental retardation is both a symptom of other disorders as well as a unique syndrome or disorder.

It is emerging that mental retardation can result from a wide range of protein abnormalities. The genes identified earliest were frequently signaling molecules in the RhoGTPase pathway (GDI, PAK3, ARHGEF6) [15] or associated with chromatin remodeling (RPS6KA3, ATRX) (17-19). Most recently, parts of the synaptic vesicle or components necessary for its formation have been identified as defective (SYN1, SLC6A8, NLGN4, and DLG3), and a number of novel transcription factors (ZNF41, 81 and 674) have been found [3, 16]. The number of identified genes that cause mental retardation suggests that a mental retardation phenotype can emerge as the final common pathway of many different types of abnormal cellular processing and that no one dominant mechanism is likely to be the cause of mental retardation. Although these observations are a far way from suggesting therapeutic treatments in humans, the possibility of offering drugs to treat some aspects of learning disability is now a realistic possibility in the future. The original aim to understand the molecular basis of intellectual disability and to provide better clinical service to this group of patients and their families is therefore rapidly being achieved.

The next challenge is to provide cheap, accurate, and effective mutation analysis for families with a history of mental retardation. Together with this, rapid and effective functional assessments of pathogenicity of variants for all genes need to be developed. The development and proper assessment of animal models of disease is needed in order to understand the pathways and improve the chances of finding therapeutic targets for learning and memory disabilities in patients in the future.

It is estimated that the lifetime costs for all people with mental retardation who were born in 2000 will total $51.2 billion (in 2003 dollars) [17, 18]. The average lifetime cost for one person with mental retardation is estimated to be $1,014,000 (in 2003 dollars). This represents costs over and above those experienced by a person who does not have a disability.

Prader-Willi syndrome (PWS) is a developmental disorder characterized by mental retardation (MR), infantile hypotonia, poor suck reflex, growth retardation, and childhood onset of pronounced hyperphagia resulting in morbid obesity [19-21]. PWS is a classic imprinting disorder with most cases resulting from paternal deletions of 15q11-q13 or maternal uniparental disomy 15 [22, 23]. However, not all patients who present with PWS-like phenotype have chromosome 15 involvement, suggesting genetic heterogeneity. Recently, a PWS-like phenotype has also been described in patients with chromosome abnormalities involving the chromosome region 6q16.2 that includes the SIM1 gene [24]. Cytogenetic and gene studies, including a screening for the SIM1 gene deletion, have recently been performed on 87 patients with a PWS-like phenotype. These observations suggest that mutational analysis and further studies of the parental origin of chromosome alterations of 6q16.2 in patients with and without a PWS-like phenotype are needed to evaluate possible imprinting effects of the SIM1 gene and establish the contribution that alterations in this gene make to the etiology of syndromic and non-syndromic obesity [24, 25].

WD-repeat proteins are defined by the presence of four or more repeating units containing a conserved core of approximately 40 amino acids that usually end with tryptophan-aspartic acid (WD) [26]. WD-repeat proteins belong to a large and fast-expanding conservative protein family. The G protein was the first WD-repeat protein to be crystallized. The crystal structure of the G protein β subunit showed a β propeller structure. All WD-repeat proteins are believed to form a circularized β propeller structure [27, 28]. The importance of these proteins is underscored by the critical roles they play in many essential biological functions ranging from signal transduction to transcription regulation and apoptosis.

There is a recognized association of WD-repeat proteins with several human diseases, such as Lissencephaly-1, Cockayne syndrome, and Allgrove syndrome. The Lissencephaly-1 gene (LIS1) was the first gene encoding a WD-repeat protein that had a proven association with a human disease. Both deletion and point mutations in the LIS1 gene are linked to Lissencephaly [29, 30]. Cockayne syndrome (CS) is associated with mutations in two genes, CSA and CSB. The CSA gene has five WD repeats, and missense and deletion mutations in this gene are associated with CS [31, 32]. Triple-A syndrome, which is also known as Allgrove syndrome, is associated with mutations in the AAAS gene [33]. X-linked ocular albinism is caused by a deletion and point mutation in the OA1 gene, which contains WD repeats [34].

WD-repeat proteins are evolutionarily conserved and have diverse functions. WD-repeat proteins perform a broad variety of cellular functions [35-37]. Currently, a total of 136 WD repeat-containing proteins have been identified in the human genome [38]. Most of these proteins seem to be regulatory. A recent study has shown that over 30 functional subfamilies can be identified among the current WD-repeat-containing proteins observed in sequenced genomes [38]. These subfamilies are roughly categorized into signal transduction, RNA synthesis/processing, chromatin assembly, vesicular trafficking, cytoskeletal assembly, cell cycle control, apoptosis, and unknown [39-42].

Sequencing of the human genome has led to a major revelation; namely, the human genome seems to encode far fewer genes (approximately 30,000) than the commonly established prediction (50,000 to over 140,000) [43, 44]. The number of genes encoded by the human genome is only about twice as many as those found in the worm (Caenorhabditis elegans) and the fly (Drosophila melanogaster). As a consequence, it appears that gene and protein networks, as opposed to simple gene number, must be responsible for the complex molecular processes of human development and physiology. Alternative mRNA splicing, RNA editing, and post-translational protein modification have been suggested as contributing mechanisms. Another critical contributing source derives from protein-protein interactions, in which WD-repeat proteins are central players. To date, many WD-repeat proteins have been identified and their associated function defined, whereas for others, the function remains unknown. The functions identified to date range from signal transduction to cell cycle control. The importance of this family of proteins is evident; their sequence has been conserved across all species in eukaryotes, these proteins perform multiple essential functions, and several human diseases have been recognized as resulting from mutations in WD-repeat proteins.

Thus, the present invention overcomes previous shortcomings in the art by providing methods and compositions for detection of mutations in the DMXL1 gene and/or the DMXL2 gene and the identification of subjects having an increased likelihood of having DMXL-associated mental retardation and/or of having DMXL-associated mental retardation.

### SUMMARY OF THE INVENTION

In one aspect, the present invention provides a method of identifying a human subject as having an increased likelihood of having DMXL-associated mental retardation, comprising detecting, in a nucleic acid sample from the subject, a mutation in a nucleotide sequence encoding DMXL1 and/or a mutation in a nucleotide sequence encoding DMXL2.

In an additional aspect, the present invention provides a method of identifying a human subject as having DMXL-associated mental retardation, comprising detecting, in a nucleic acid sample from the subject, a mutation in a nucleotide sequence encoding DMXL1 and/or a mutation in a nucleotide sequence encoding DMXL2.

Further provided herein is a method of screening a human subject for DMXL-associated mental retardation, comprising detecting, in a nucleic acid sample from the subject, a mutation in a nucleotide sequence encoding DMXL1 and/or a mutation in a nucleotide sequence encoding DMXL2.

In yet further aspects, the present invention provides a method of identifying a human subject as having an increased likelihood of having DMXL-associated mental retardation, comprising detecting, in a sample from the subject, a mutation in a DMXL1 protein and/or a mutation in a DMXL2 protein.

Additionally provided is a method of identifying a human subject as having DMXL-associated mental retardation, comprising detecting, in a sample from the subject, a mutation in a DMXL1 protein and/or a mutation in a DMXL2 protein.

Furthermore, the present invention provides a method of screening a human subject for DMXL-associated mental retardation, comprising detecting, in a sample from the subject, a mutation in a DMXL1 protein and/or a mutation in a DMXL2 protein.

Also provided herein is a method of amplifying a segment of a nucleotide sequence encoding DMXL1, comprising: a) choosing a first oligonucleotide primer from a nucleic acid sequence comprising the nucleotide sequence of SEQ ID NO:1, the nucleotide sequence of SEQ ID NO:3 or the nucleotide sequence of SEQ ID NO:5; b) choosing a second oligonucleotide primer from a nucleic acid sequence comprising the nucleotide sequence of SEQ ID NO:1, the nucleotide sequence of SEQ ID NO:3 or the nucleotide sequence of SEQ ID NO:5 that differs in nucleotide sequence from the first oligonucleotide primer; c) adding said first oligonucleotide primer and said second oligonucleotide primer to a nucleic acid sample; and d) amplifying a segment of the nucleotide sequence encoding DMXL1 defined by said first oligonucleotide primer and said second oligonucleotide primer, wherein said nucleic acid sample is from a subject having a DMXL-associated mental retardation phenotype.

Also provided herein is a method of amplifying a segment of a nucleotide sequence encoding DMXL2, comprising: a) choosing a first oligonucleotide primer from a nucleic acid sequence comprising the nucleotide sequence of SEQ ID NO:2, the nucleotide sequence of SEQ ID NO:4 or the nucleotide sequence of SEQ ID NO:7; b) choosing a second oligonucleotide primer from a nucleic acid sequence comprising the nucleotide sequence of SEQ ID NO:2, the nucleotide sequence of SEQ ID NO:4 or the nucleotide sequence of SEQ ID NO:7 that differs in nucleotide sequence from the first oligonucleotide primer; c) adding said first oligonucleotide primer and said second oligonucleotide primer to a nucleic acid sample; and d) amplifying a segment of the nucleotide sequence encoding DMXL2 defined by said first oligonucleotide primer and said second oligonucleotide primer, wherein said nucleic acid sample is from a subject having a DMXL-associated mental retardation phenotype.

Other and further objects, features and advantages would be apparent and more readily understood by reading the following specification and by reference to the accompanying drawing forming a part thereof, or any examples of the embodiments of the invention given for the purpose of the disclosure.

### BRIEF DESCRIPTION OF DRAWINGS

FIG. 1 shows the locations, relative to the exon involved, of some of the nucleotide sequence mutations identified in the DMXL1 gene, which resulted in the corresponding amino acid mutations detected in DMXL.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

The present invention is based on the unexpected discovery that a mutation in the DMXL1 gene and/or DMXL2 gene of a subject, particularly a mutation that disrupts the function of DMXL1 and/or DMXL2, results in the phenotype described herein and identifies a subject as having an increased likelihood of having DMXL-associated mental retardation or of having DMXL-associated mental retardation. Thus, when a subject demonstrating the phenotype of this invention is tested and a mutation in a nucleic acid of the subject in the DMXL1 gene and/or DMXL2 gene is detected, the subject is identified as having or is identified as having an increased likelihood of having what is defined herein as “DMXL-associated mental retardation.” It has been the inventors' observation that any mutation in the DMXL1 gene and/or DMXL2 gene, particularly a mutation that disrupts the function of DMXL1 and/or DMXL2, in a subject demonstrating the phenotype of this invention allows for the identification of a subject as having DMXL-associate mental retardation or as having an increased likelihood of having DMXL-associated mental retardation.

Thus, the invention is the discovery of the association between a function-disrupting mutation of the DMXL1 gene and/or DMXL2 gene and the phenotype of this invention. Although several exemplary mutations are described herein, the specific mutation or mutations detected do not define the invention; rather the invention is defined by the presence of any mutation in the DMXL1 gene and/or DMXL2 gene and its association with the phenotype described herein. The detection of any mutation in the DMXL1 gene and/or DMXL2 gene of a subject having the phenotype described herein allows for the identification of that subject as having DMXL-associated mental retardation or as having an increased likelihood of having DMXL-associated mental retardation.

As noted herein, many subjects of the studies described herein who present with clinical features that resemble other neurodevelopmental disorders have tested negative for known neurodevelopmental disorders such as Prader-Willi syndrome and/or Rett syndrome.

The present invention allows for a clinician to assign a diagnosis (either by the methods of this invention alone and/or in combination with other information known to one of skill in the art) to such subjects having the phenotype of this invention and a function-disrupting mutation in the DMXL1 gene and/or DMXL2 gene, which is invaluable to the clinician and medical community in developing treatment plans as well as in monitoring and documenting any changing/evolving aspects and/or unique characteristics of the phenotype, in order to understand and subsequently identify ways to prevent, treat and/or cure DMXL-associated mental retardation.

Such identification is also extremely valuable to family members, relatives, educators, social workers and caregivers, in allowing for an understanding of the genetic nature of the phenotype as well as an understanding of the subject's clinical features and symptoms, which facilitates addressing the medical as well as the social, emotional, educational and developmental needs of the subject.

Furthermore, this discovery facilitates development of a test for early diagnosis and prenatal detection of DMXL-associated mental retardation. Even more importantly, the discovery of the association of a mutation in the DMXL1 gene and/or DMXL2 gene and the neuropathological phenotype described herein identifies the genetic basis for this disorder, providing opportunities for therapy.

Thus in one embodiment, the present invention provides a method of identifying a human subject as having an increased likelihood of having DMXL-associated mental retardation, comprising detecting, in a nucleic acid sample from the subject, a mutation in a nucleotide sequence encoding DMXL1 and/or a mutation in a nucleotide sequence encoding DMXL2.

Further provided herein is a method of identifying a human subject as having DMXL-associated mental retardation, comprising detecting, in a nucleic acid sample from the subject, a mutation in a nucleotide sequence encoding DMXL1 and/or a mutation in a nucleotide sequence encoding DMXL2.

The present invention further provides a method of screening a human subject for DMXL-associated mental retardation, comprising detecting, in a nucleic acid sample from the subject, a mutation in a nucleotide sequence encoding DMXL1 and/or a mutation in a nucleotide sequence encoding DMXL2.

In additional embodiments, the present invention provides a method of identifying a human subject as having an increased likelihood of having DMXL-associated mental retardation, comprising detecting, in a sample from the subject, a mutation in a DMXL1 protein and/or a mutation in a DMXL2 protein.

Furthermore, the present invention provides a method of identifying a human subject as having DMXL-associated mental retardation, comprising detecting, in a sample from the subject, a mutation in a DMXL1 protein and/or a mutation in a DMXL2 protein.

Additionally provided herein is a method of screening a human subject for DMXL-associated mental retardation, comprising detecting, in a sample from the subject, a mutation in a DMXL1 protein and/or a mutation in a DMXL2 protein.

Exemplary mutations of this invention include, but are not limited to, the following nucleotide substitutions (with numbering based on the nucleotide sequence of SEQ ID NO:5 for DMXL1 and the nucleotide sequence of SEQ ID NO:7 for DMXL2): 5476C>T in the nucleotide sequence encoding DMXL1, 5947A>G in the nucleotide sequence encoding DMXL1, 6166 C>T in the nucleotide sequence encoding DMXL1, 1462 A>G in the nucleotide sequence encoding DMXL1, 3716 G>C in the nucleotide sequence encoding DMXL1, 4765A>G in the nucleotide sequence encoding DMXL1, 5321 G>A in the nucleotide sequence encoding DMXL1, 6199G>A in the nucleotide sequence encoding DMXL1, 6225C>G in the nucleotide sequence encoding DMXL1, 6839G>T in the nucleotide sequence encoding DMXL1, 8198G>A in the nucleotide sequence encoding DMXL1, 4466G>T in the nucleotide sequence encoding DMXL2, 6032A>T in the nucleotide sequence encoding DMXL2 and any combination thereof.

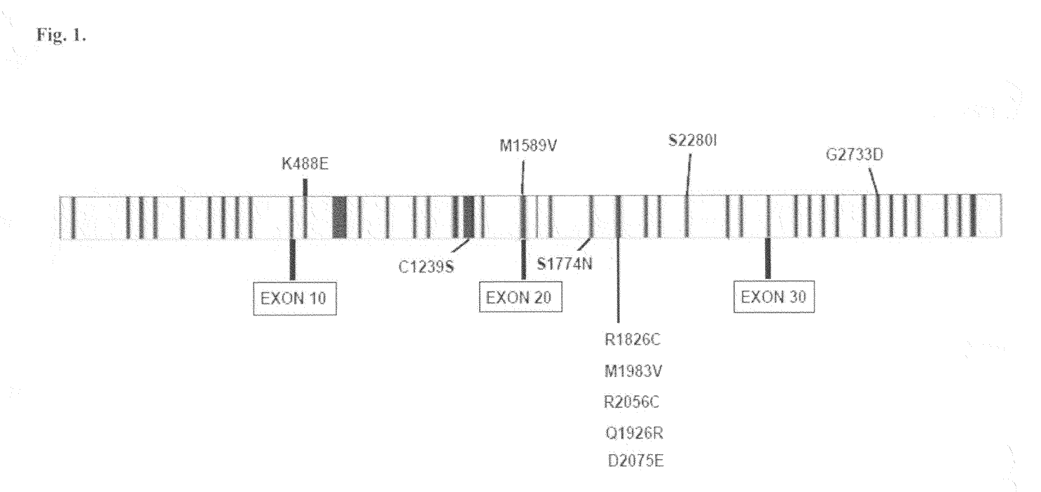

At the amino acid level, an exemplary mutation of this invention can be an amino acid sequence mutation having the following amino acid substitutions (with numbering based on the amino acid sequence of SEQ ID NO:2 for DMXL1 and on the amino acid sequence of SEQ ID NO:4 for DMXL2): R1826C in DMXL1, M1983V in DMXL1, R2056C in DMXL1, K488E in DMXL1, C1239S in DMXL1, M1589V in DMXL1, S1774N in DMXL1, V2067M in DMXL1, D2075E in DMXL1, G2733D in DMXL1, Q1926R in DMXL1, R1489I in DMXL2, D2011V in DMXL2 and any combination thereof. The positions of the DMXL1 protein mutations relative to the location of the corresponding nucleotide sequence mutations are shown in FIG. 1.

As noted above, the invention is not limited to these exemplary mutations and encompasses the detection of previously undetected mutations according to the methods described herein in order to identify a subject as having an increased likelihood of having DMXL-associated mental retardation or as having DMXL-associated mental retardation.

In the methods of this invention, the mutation in the DMXL1 gene and/or DMXL2 gene can be missense mutation. In further embodiments, the mutation can be a nonsense, a missense, a frameshift, an insertion or a deletion of one or more base pairs. Mutations could lead to a truncated protein, could alter the conformation of the protein or could directly affect an amino acid required for function of the protein. A mutation that produces no deleterious effects on, or disruption of, the function or structure of the DMXL1 protein and/or the DMXL2 protein and produces no detectable phenotype is not included in the present invention. A mutation is detected relative to the wild type sequence of the DMXL1 gene (SEQ ID NO:1), DMXL2 gene (SEQ ID NO:2), DMXL1 mRNA (SEQ ID NO:3). DMXL2 mRNA (SEQ ID NO:4), DMXL1 coding sequence (SEQ ID NO:5), DMXL2 coding sequence (SEQ ID NO:7), DMXL1 amino acid sequence (SEQ ID NO:6) and DMXL2 amino acid sequence (SEQ ID NO:8), as set forth in the attached Sequence Listing.

In some embodiments of this invention, the mutation in the DMXL1 gene and/or DMXL2 gene is in a noncoding region of the gene and/or in a coding region of the gene. For example, regions of a nucleotide sequence encoding DMXL1 and/or DMXL2 that can be analyzed (e.g., by amplification) to detect a mutation include regulatory regions (e.g., promoter sequence, enhancer sequence, termination sequence, cis-acting sequence, etc.), exons, introns, exon/intron junctions, untranslated regions, etc. as are well known in the art. The nucleic acid can be employed in the methods of this invention as genomic nucleic acid, mRNA, cDNA or any combination thereof.

In the methods of this invention, a mutation in a nucleotide sequence encoding DMXL1 and/or in a nucleotide sequence encoding DMXL2 can be detected by a variety of methods well known in the art for analyzing nucleic acid. Such methods include but are not limited to sequencing, electrophoresis, nucleic acid hybridization (e.g., with hybridization probe(s) attached to a solid support), fluorescence in situ hybridization, reverse transcription-amplification (e.g., polymerase chain reaction, ligase chain reaction, etc.), high-performance liquid chromatography (HPLC) (e.g., denaturing HPLC), restriction fragment length polymorphism analysis, comparative genomic hybridization (CGH) array and any combination thereof.

Furthermore, in the methods of this invention, a mutation in an amino acid sequence of DMXL1 and/or in an amino acid sequence of DMXL2 can be detected by a variety of methods well known in the art for analyzing proteins and amino acid sequences. Such methods include but are not limited to sequencing, electrophoresis, immunoassay, molecular weight analysis, mRNA profiling and any combination thereof.

In some embodiments of this invention, the subject has a phenotype of clinical features that demonstrate that the subject has a neurodevelopmental disorder. This phenotype, referred to herein as the phenotype of this invention and as a “DMXL-associated mental retardation phenotype” is defined as a collection of clinical features comprising, consisting essentially of, or consisting of microcephaly (e.g., acquired microcephaly), hypotonia transitioning to spasticity in childhood and language impairment (e.g., severe language impairment). Other clinical features of this phenotype can comprise, consist essentially of, or consist of mental retardation, poor suck reflex, growth retardation, strabismus, hyperactivity, later onset obesity, prominent forehead, low-set, dysplastic ears, palatal anomalies, down-turned corners of the mouth, receding chin, small hands/feet, tapered fingers, camptodactyly and/or genital malformations, in any combination. In some embodiments, one or more of these clinical features is not detected in the subject and therefore the phenotype can be defined as the absence of said one or more clinical features.

In addition or alternatively, a subject of this invention can also be a subject that has tested negative for one or more neurodevelopmental disorders and/or can be a subject who does not meet the criteria for clinical diagnosis of one or more neurodevelopmental disorders. Such neurodevelopmental disorders include but are not limited to Prader Willi syndrome, Rett syndrome, Angelman syndrome, autism, non-syndromic mental retardation, idiopathic neonatal encephalopathy, idiopathic infantile spasms, idiopathic cerebral palsy, schizophrenia, Fragile X syndrome, Down syndrome, Cri-du-chat syndrome, Rubenstein-Taybi syndrome, fetal alcohol syndrome and any combination thereof.

A subject of this invention that has the phenotype of this invention and/or who has tested negative for a neurodevelopmental disorder for which testing is available and/or who does not meet the criteria for clinical diagnosis for a specific neurodevelopmental disorder can be tested for the presence of a mutation in the subject's DMXL1 gene and/or DMXL2 gene according to the methods described herein. Such a subject in whom such a mutation is detected is a subject having an increased likelihood of having DMXL-associated mental retardation or is a subject having DMXL-associated mental retardation. As used herein, the term “DMXL-associated mental retardation” is a disorder defined by the presence of the DMXL-associated mental retardation phenotype of this invention in a subject and a mutation in the DMXL1 gene and/or DMXL2 gene of the subject.

In some embodiments of this invention, the mutation in the DMXL1 gene and/or DMXL2 gene results in disruption of the function of the DMXL1 protein and/or the DMXL2 protein. Disruption of the function of the DMXL1 protein and/or the DMXL2 protein can result in the development of the phenotype of this invention in a subject.

The terms “disruption of the function” or “disrupts function” as used herein are defined as a prohibition or interference with the normal function of the DMXL1 protein and/or the DMXL2 protein. In another embodiment, the terms refer to prohibiting or interfering with normal function of a complex of proteins that includes the DMXL1 protein and/or the DMXL2 protein.

Methods of determining if the function of a DMXL1 protein and/or a DMXL2 protein include, but are not limited to, measuring the amount of mRNA encoding DMXL1 protein and/or a DMXL2 protein in a sample from a subject and/or measuring the amount of a DMXL1 protein and/or a DMXL2 protein in a sample from a subject, wherein if the amount of mRNA and/or protein is decreased relative to a control, a determination can be made that the function of the DMXL1 protein and/or DMXL2 protein is disrupted.

Other methods of determining if the function of DMXL1 protein and/or DMXL2 protein has been disrupted include measuring the amount of activity of DMXL1 protein and/or DMXL2 protein in a sample from a subject. For example, the amount of activity of a DMXL1 protein and/or a DMXL2 protein can be determined by carrying vesicular trafficking assays according to methods well known in the art and as described herein. A modulation (e.g., an increase or decrease) in the amount of activity of a DMXL1 protein and/or a DMXL2 protein relative to a control allows for a determination that the function of the DMXL1 protein and/or DMXL2 protein has been disrupted.

The present invention further provides methods of amplifying nucleic acid in a subject having the phenotype of this invention. Thus, in one embodiment, the present invention provides a method of amplifying a segment of a nucleotide sequence encoding DMXL1, comprising: a) choosing a first oligonucleotide primer from a nucleic acid sequence comprising, consisting essentially of or consisting of the nucleotide sequence of SEQ ID NO:1, the nucleotide sequence of SEQ ID NO:3 or the nucleotide sequence of SEQ ID NO:5; b) choosing a second oligonucleotide primer from a nucleic acid sequence comprising, consisting essentially of or consisting of the nucleotide sequence of SEQ ID NO:1, the nucleotide sequence of SEQ ID NO:3 or the nucleotide sequence of SEQ ID NO:5 that differs in nucleotide sequence from the first oligonucleotide primer; c) adding said first oligonucleotide primer and said second oligonucleotide primer to a nucleic acid sample; and d) amplifying a segment of the nucleotide sequence encoding DMXL1 defined by said first oligonucleotide primer and said second oligonucleotide primer, wherein said nucleic acid sample is from a subject having a phenotype of this invention (e.g., a DMXL-associated mental retardation phenotype).

Further provided herein is a method of amplifying a segment of a nucleotide sequence encoding DMXL2, comprising: a) choosing a first oligonucleotide primer from a nucleic acid sequence comprising, consisting essentially of, or consisting of the nucleotide sequence of SEQ ID NO:2, the nucleotide sequence of SEQ ID NO:4 or the nucleotide sequence of SEQ ID NO:7; b) choosing a second oligonucleotide primer from a nucleic acid sequence comprising, consisting essentially of, or consisting of the nucleotide sequence of SEQ ID NO:2, the nucleotide sequence of SEQ ID NO:4 or the nucleotide sequence of SEQ ID NO:7 that differs in nucleotide sequence from the first oligonucleotide primer; c) adding said first oligonucleotide primer and said second oligonucleotide primer to a nucleic acid sample; and d) amplifying a segment of the nucleotide sequence encoding DMXL2 defined by said first oligonucleotide primer and said second oligonucleotide primer, wherein said nucleic acid sample is from a subject having a phenotype of this invention (e.g. a DMXL-associated mental retardation phenotype).

In the amplification methods of this invention, the amplified segment of step (d) above can be about 50, about 100, about 150, about 200, about 250, about 300, about 400, about 500 or about 1000 base pairs in length. Furthermore, the amplified segment can comprise a mutation that disrupts the function of the DMXL1 protein and/or DMXL2 protein. In some embodiments, the primers can be designed according to methods well known in the art to lead to the production of an amplification product only if a mutation is present or only if no mutation is present. The amplification product can be identified, quantitated and/or characterized by methods standard in the art (e.g., hybridization, restriction endonuclease analysis, sequencing, etc.)

In some embodiments, the first oligonucleotide primer can be at least about 15, 20, 25, 30, 35, 40, 45, 50, 60, 70, 80, 90 or 100 nucleotides in length. In some embodiments, the second oligonucleotide primer can be at least about 15, 20, 25, 30, 35, 40, 45, 50, 60, 70, 80, 90 or 100 nucleotides in length.

In further aspects, the present invention provides a kit for carrying out the methods of this invention, wherein the kit can comprise primers, probes, primer/probe sets, reagents, buffers, etc., as would be known in the art, for the detection of mutation in the Dmxl1 gene and/or the Dmxl2 gene in a nucleic acid sample from the subject and/or for the detection of mutation in the DMXL1 protein and/or DMXL2 protein in a sample from a subject. For example, a primer or probe can comprise a contiguous nucleotide sequence that is complementary (or sufficiently complementary) to a nucleotide sequence encoding a DMXL1 protein and/or a nucleotide sequence encoding a DMXL2 protein. Such a kit can further comprise blocking probes, labeling reagents, blocking agents, restriction enzymes, antibodies (e.g., secondary antibodies), ligands, immunoglobulin binding agents, sampling devices, positive and negative controls, etc., as would be well known to those of ordinary skill in the art.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The term “chromosome region” as used herein refers to a part of a chromosome defined either by anatomical details, especially by banding, or by its linkage groups. The particular chromosome regions of this invention are further defined by the following boundaries.

Also as used herein, “linked” describes a region of a chromosome that is shared more frequently in family members or members of a population manifesting a particular phenotype and/or affected by a particular disease or disorder, than would be expected or observed by chance, thereby indicating that the gene or genes or other identified marker(s) within the linked chromosome region contain or are associated with an allele that is correlated with the phenotype and/or presence of a disease or disorder, or with an increased or decreased likelihood of the phenotype and/or of the disease or disorder. Once linkage is established, association studies (linkage disequilibrium) can be used to narrow the region of interest or to identify the marker (e.g., allele or haplotype) correlated with the phenotype and/or disease or disorder.

Furthermore, as used herein, the term “linkage disequilibrium” or “LD” refers to the occurrence in a population of two linked alleles at a frequency higher or lower than expected on the basis of the gene frequencies of the individual genes. Thus, linkage disequilibrium describes a situation where alleles occur together more often than can be accounted for by chance, which indicates that the two alleles are physically close on a DNA strand.

The term “genetic marker” or “polymorphism” as used herein refers to a characteristic of a nucleotide sequence (e.g., in a chromosome) that is identifiable due to its variability among different subjects (i.e., the genetic marker or polymorphism can be a single nucleotide polymorphism, a restriction fragment length polymorphism, a microsatellite, a deletion of nucleotides, an addition of nucleotides, a substitution of nucleotides, a repeat or duplication of nucleotides, a translocation of nucleotides, and/or an aberrant or alternate splice site resulting in production of a truncated or extended form of a protein, etc., as would be well known to one of ordinary skill in the art).

A “single nucleotide polymorphism” (SNP) in a nucleotide sequence is a genetic marker that is polymorphic for two (or in some case three or four) alleles. SNPs can be present within a coding sequence of a gene, within noncoding regions of a gene and/or in an intergenic (e.g., intron) region of a gene. A SNP in a coding region in which both forms lead to the same polypeptide sequence is termed synonymous (i.e., a silent mutation) and if a different polypeptide sequence is produced, the alleles of that SNP are non-synonymous. SNPs that are not in protein coding regions can still have effects on gene splicing, transcription factor binding and/or the sequence of non-coding RNA.

The SNP nomenclature provided herein refers to the official Reference SNP (rs) identification number as assigned to each unique SNP by the National Center for Biotechnological Information (NCBI), which is available in the GenBank® database.

In some embodiments, the term genetic marker is also intended to describe a phenotypic effect of an allele or haplotype, including for example, an increased or decreased amount of a messenger RNA, an increased or decreased amount of protein, an increase or decrease in the copy number of a gene, production of a defective protein, tissue or organ, etc., as would be well known to one of ordinary skill in the art.

An “allele” as used herein refers to one of two or more alternative forms of a nucleotide sequence at a given position (locus) on a chromosome. Usually alleles are nucleotides present in a nucleotide sequence that makes up the coding sequence of a gene, but sometimes the term is used to refer to a nucleotide in a non-coding region of a gene. An individual's genotype for a given gene is the set of alleles it happens to possess. As noted herein, an individual can be heterozygous or homozygous for an allele of this invention.

Also as used herein, a “haplotype” is a set of SNPs on a single chromatid that are statistically associated. It is thought that these associations, and the identification of a few alleles of a haplotype block, can unambiguously identify all other polymorphic sites in its region. The term “haplotype” is also commonly used to describe the genetic constitution of individuals with respect to one member of a pair of allelic genes; sets of single alleles or closely linked genes that tend to be inherited together.

The terms “increased likelihood” or “increased risk” as used herein define the likelihood or the level of risk that a subject has of having or developing DMXL-associated mental retardation, as compared to a control subject that does not have a mutation in the Dmxl1 gene or the Dmxl2 gene.

The term “antisense” as used herein is defined as the sequence of a gene which is complementary to the sequence of the gene which encodes the gene product.

The term “exon” as used herein is defined as a transcribed segment of a gene that is present in a mature messenger RNA molecule.

The term “exon/intron junction” as used herein is defined as two specific nucleotide locations at which point an intronic sequence is spliced from an RNA transcript.

The term “idiopathic” as used herein is defined as of unknown cause.

The term “intron” as used herein is defined as a region of a gene transcribed from a DNA template but subsequently removed by splicing together the segments (exons) which flank it.

The term “nucleic acid chip technology” as used herein is defined as the method of immobilizing nucleic acid on a microchip for subsequent hybridization analysis.

The term “pharmacologically effective dose” is the amount of an agent administered to be physiologically significant. An agent is physiologically significant if its presence results in a positive or negative change in the physiology of a recipient mammal.

The term “polymerase chain reaction” (PCR) is well known in the art and includes the method of amplifying a nucleic acid sequence utilizing two oligonucleotide primers and a thermolabile nucleic acid polymerase.

The term “reverse transcription-polymerase chain reaction” as used herein is defined as the polymerization of a DNA molecule using an RNA molecule as a template for the purpose of utilizing said DNA molecule as a template for PCR.

The term “splicing” as used herein is defined as a means of removing intron sequences within a primary RNA transcript in processing of said transcript to a mature messenger RNA.

The term “suicide gene” as used herein is defined as a gene whose gene product is lethal to a cell upon exposure to a prodrug.

The term “therapeutically effective” as used herein is defined as the amount of a compound required to improve some symptom associated with a disease. For example, in the treatment of neurodevelopmental disease, a compound which decreases, prevents, delays, or arrests any symptom of the disease would be therapeutically effective. A therapeutically effective amount of a compound is not required to cure a disease but will provide a treatment for a disease.

The term 3′ untranslated region (3′ UTR) as used herein is defined as the sequence at the 3′ end of a messenger RNA that does not become translated into protein and can include regulatory sequences and sequences important for posttranscriptional processing.

The term “transcribe” as used herein is defined as the process of generating an RNA transcript molecule using DNA as a template.

The term “transcript” as used herein is defined as an RNA molecule which has been transcribed from DNA.

A sample of this invention can be any sample containing nucleic acid and/or protein of a subject, as would be well known to one of ordinary skill in the art. Nonlimiting examples of a sample of this invention include a cell, a body fluid, a tissue, a washing, a swabbing, etc., as would be well known in the art. Nonlimiting examples include white blood cells, saliva and skin cells.

A subject of this invention is any animal that carries a DMXL1 gene and/or DMXL2 gene as well as animal models (e.g., rats, mice, dogs, nonhuman primates, etc.). In some aspects of this invention, the subject can be a human of any ethnicity or race (e.g., Caucasian; white; European, European-American; Hispanic; Latin-American; black; African; African American; African-European; African-Caribbean; African-Australian; African-Asiatic; Austronesian; Mideastern; Asian; Polynesian; Inuit; etc., as are well known in the art). Furthermore, a subject of this invention can be a fetus, a neonate, an infant, a child, an adolescent, a teenager or an adult. Thus, in some embodiments, it is contemplated that the methods of this invention can be used for prenatal testing of a fetus, as well as for testing of a subject of any age.

In further aspects of this invention, the subject has a family history of mutation in the DMXL1 gene and/or DMXL2 gene and in some embodiments, the subject does not have a family history of DMXL1 gene and/or DMXL2 gene (i.e., the mutation in the subject is a de novo mutation).

As used herein, “nucleic acid” encompasses both RNA and DNA, including cDNA, genomic DNA, mRNA, synthetic (e.g., chemically synthesized) DNA and chimeras, fusions and/or hybrids of RNA and DNA. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides, etc.). Such oligonucleotides can be used, for example, to prepare nucleic acids that have altered base-pairing abilities or increased resistance to nucleases.

An “isolated nucleic acid” is a nucleotide sequence that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence.

The term “isolated” can refer to a nucleic acid or polypeptide that is substantially free of cellular material, viral material, and/or culture medium (e.g., when produced by recombinant DNA techniques), or chemical precursors or other chemicals (when chemically synthesized). Moreover, an “isolated fragment” is a fragment of a nucleic acid or polypeptide that is not naturally occurring as a fragment and would not be found in the natural state.

The term “oligonucleotide” refers to a nucleic acid sequence of at least about six nucleotides to about 100 nucleotides, for example, about 15 to about 30 nucleotides, or about 20 to about 25 nucleotides, which can be used, for example, as a primer in a PCR amplification and/or as a probe in a hybridization assay or in a microarray. Oligonucleotides of this invention can be natural or synthetic, e.g., DNA, RNA, PNA, LNA, modified backbones, etc., as are well known in the art.

The present invention further provides fragments of the nucleic acids of this invention, which can be used, for example, as primers and/or probes. Such fragments or oligonucleotides can be detectably labeled or modified, for example, to include and/or incorporate a restriction enzyme cleavage site when employed as a primer in an amplification (e.g., PCR) assay.

The detection of a mutation of this invention can be carried out according to various protocols standard in the art and as described herein for analyzing nucleic acid samples and nucleotide sequences, as well as identifying specific nucleotides in a nucleotide sequence.

For example, nucleic acid can be obtained from any suitable sample from the subject that will contain nucleic acid and the nucleic acid can then be prepared and analyzed according to well-established protocols for the presence of mutations according to the methods of this invention. In some embodiments, analysis of the nucleic acid can be carried by direct sequencing of the nucleotide sequence encoding DMXL1 and/or DMXL2 (e.g., from a coding sequence, a messenger RNA sequence and/or a genomic sequence, including but not limited to a regulatory sequence [e.g., an enhancer sequence, a promoter sequence, a cis sequence that binds transacting factors, etc.), an exon, an intron, an exon/intron junction, a 5′ or 3′ untranslated region and any combination thereof].

In some embodiments, analysis of the nucleic acid can be carried out by amplification of the region of interest according to amplification protocols well known in the art (e.g., polymerase chain reaction, ligase chain reaction, strand displacement amplification, transcription-based amplification, self-sustained sequence replication (3SR), Qβ replicase protocols, nucleic acid sequence-based amplification (NASBA), repair chain reaction (RCR) and boomerang DNA amplification (BDA), etc.). The amplification product can then be visualized directly in a gel by staining or the product can be detected by hybridization with a detectable probe. When amplification conditions allow for amplification of all allelic types of a genetic marker, the types can be distinguished by a variety of well-known methods, such as hybridization with an allele-specific probe, secondary amplification with allele-specific primers, by restriction endonuclease digestion, and/or by electrophoresis. Thus, the present invention further provides oligonucleotides for use as primers and/or probes for detecting and/or identifying genetic markers according to the methods of this invention.

Mutations in nucleic acid sequences which encode DMXL1 and/or DMXL2 proteins can be detected in a variety of methods known to those in the art including by sequencing, probe, nucleic acid hybridization, PCR, nucleic acid chip hybridization, electrophoresis, or fluorescent in situ hybridization (FISH). Sequencing methods are common laboratory procedures known to many in the art and would be able to detect the exact nature of the mutation. In addition, mutation could be detected by probe. For instance, one skilled in the art would be aware that a fluorescent tag could be specific for binding of a mutation and could be exposed to, for instance, glass beads coated with nucleic acids containing potential mutations. Upon binding of the tag to the mutation in question, a change in fluorescence (such as creation of fluorescence, increase in intensity, or partial or complete quenching) could be indicative of the presence of that mutation. Nucleic acid hybridization including. Southern or Northern blot assays could be utilized to detect mutations such as those involved in alteration of large regions of the sequence or of those involved in alteration of a sequence containing a restriction endonuclease site.

Hybridization is detected by a variety of ways including radioactivity, color change, light emission, or fluorescence. PCR could also be used to amplify a region suspected to contain a mutation and the resulting amplified region could either be subjected to sequencing or to restriction digestion analysis in the event that mutation was responsible for creating or removing a restriction endonuclease site. The mutation could be identified through an RNA species from the gene by RT-PCR methods which are well known in the art.

One skilled in the art would also know that a specific method of nucleic acid hybridization could be utilized in the form of nucleic acid chip hybridization in which nucleic acids are present on a immobilized surface such as a microchip or microchips and are subjected to hybridization techniques sensitive enough to detect minor changes in sequences; a variety of detection methods could be used including light emission, fluorescence, color change, or radioactivity. Electrophoresis could be employed to detect mutations of the sequence either by mobility changes or in conjunction with another method of detecting a mutation such as with sequencing or by PCR. Finally, one skilled in the art would be aware that FISH is a proficient technique for detecting large regions of sequences on chromosomes which have been deleted or rearranged.

In some embodiments, he use of a probe or primer of between 13 and 100 nucleotides, between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02M to about 0.10M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25M NaCl at temperatures of about 37° C. to about 55° C., while a low stringency condition could be provided by about 0.15M to about 0.9M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mMMgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In some embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids encoding DXML1 and/or DMXL2 are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids that contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescence label or even via a system using electrical and/or thermal impulse signals (e.g., Affymax technology).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, each of which is incorporated herein by reference in its entirety.

A reverse transcriptase PCR™ amplification procedure may be performed to quantify the amount of mRNA amplified. Methods of reverse transcribing RNA into cDNA are well known and described, e.g., in Sambrook et al., 1989. Alternative methods for reverse transcription utilize thermostable DNA polymerases. These methods are described in PCT Publication No. WO 90/07641. Polymerase chain reaction methodologies are well known in the art. Representative methods of RT-PCR are described in U.S. Pat. No. 5,882,864.

Another method for amplification is ligase chain reaction (“LCR”), disclosed, e.g., in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention. Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). Davey et al., European Application No. 329 822, disclose a nucleic acid amplification process involving cyclically synthesizing single-stranded RNA (“ssRNA”), ssDNA, and double-stranded DNA (dsDNA), which may be used in accordance with the present invention.

Miller et al., PCT Publication No. WO 89/06700 (incorporated herein by reference in its entirety), discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel.

Mutations can be detected in the amino acid sequence of the DMXL1 protein and/or DMXL2 protein through the following methods: sequencing, mass spectrometry, by molecular weight, with antibodies, through increased expression of a target gene, by chromosomal coating or by alterations in methylation of DNA patterns. Examples of alterations include a change, loss, or addition of an amino acid, truncation or fragmentation of the protein. Alterations can increase degradation of the protein, can change conformation of the protein, or can be present in a hydrophobic or hydrophilic domain of the protein. The alteration need not be in an active site of the protein to have a deleterious effect on its function or structure, or both. Alteration can include modifications to the protein such as phosphorylation, myristilation, acetylation, or methylation. Sequencing of the protein or a fragment thereof directly by methods well known in the art would identify specific amino acid alterations. Alterations in protein sequences can be detected by analyzing either the entire protein or fragments of the protein and subjecting them to mass spectrometry, which would be able to detect even minor changes in molecular weight. Additionally, antibodies can be used to detect mutations in said proteins if the epitope includes the particular site that has been mutated. Antibodies can be used to detect mutations in the protein by immunoblotting, with in situ methods, or by immunoprecipitation. Antibodies to the methyl-CpG-binding domain containing protein on immunoblots may alternatively recognize any epitope of the protein and could detect truncations or modifications of the protein that would affect electrophoretic mobility, including phosphorylation or myristilation.

The term “vector” refers to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. The nucleic acid sequence can also be exogenous to the vector. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described, e.g., in Maniatis et al., 1988 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202; U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The present invention further provides a method of identifying an effective and/or appropriate (i.e., for a given subject's particular condition or status) treatment regimen for a subject with DMXL-associated mental retardation, comprising detecting a mutation in the DMXL1 gene and/or DMXL2 gene of the subject, wherein the mutation is correlated with an effective and/or appropriate treatment regimen for DMXL-associated mental retardation according to protocols as described herein and as are well known in the art.

Also provided is a method of identifying an effective and/or appropriate treatment regimen for a subject with DMXL-associated mental retardation, comprising: a) correlating the presence of one or more DMXL1 and/or DMXL2 mutations of this invention in a test subject or population of test subjects with DMXL-associated mental retardation for whom an effective and/or appropriate treatment regimen has been identified; and b) detecting the mutation(s) of step (a) in the subject, thereby identifying an effective and/or appropriate treatment regimen for the subject.

Further provided is a method of correlating a DMXL1 and/or DMXL2 mutation of this invention with an effective and/or appropriate treatment regimen for DMXL-associated mental retardation, comprising: a) detecting in a subject or a population of subjects with DMXL-associated mental retardation and for whom an effective and/or appropriate treatment regimen has been identified, the presence of one or more mutations of this invention; and b) correlating the presence of the one or more mutations of step (a) with an effective treatment regimen for DMXL-associated mental retardation.

Examples of treatment regimens for subjects having one or more of the clinical features of the phenotype of DMXL-associated mental retardation are well known in the art. Subjects who respond well to particular treatment protocols can be analyzed for specific mutations in the DMXL1 gene and/or DMXL2 gene and a correlation can be established according to the methods provided herein. Alternatively, subjects who respond poorly to a particular treatment regimen can also be analyzed for particular mutations in the DMXL1 gene and/or DMXL2 gene correlated with the poor response. Then, a subject who is a candidate for treatment for the clinical symptoms and features of DMXL-associated mental retardation can be assessed for the presence of the appropriate mutation(s) and the most effective and/or appropriate treatment regimen can be provided.

In some embodiments, the methods of correlating DMXL1 and/or DMXL2 mutations with treatment regimens of this invention can be carried out using a computer database. Thus the present invention provides a computer-assisted method of identifying a proposed treatment for DMXL-associated mental retardation. The method involves the steps of (a) storing a database of biological data for a plurality of subjects, the biological data that is being stored including for each of said plurality of subjects, for example, (i) a treatment type, (ii) at least one DMXL1 mutation associated with DMXL-associated mental retardation, and (iii) at least one clinical feature or symptom of DMXL-associated mental retardation from which treatment efficacy can be determined; and then (b) querying the database to determine the dependence on said mutation(s) of the effectiveness of a treatment type in treating DMXL-associated mental retardation, to thereby identify a proposed treatment as an effective and/or appropriate treatment for a subject having DMXL-associated mental retardation.

In one embodiment, treatment information for a subject is entered into the database (through any suitable means such as a window or text interface), DMXL1 and/or DMXL2 mutation information for that subject is entered into the database, and disease progression/clinical status information is entered into the database. These steps are then repeated until the desired number of subjects has been entered into the database. The database can then be queried to determine whether a particular treatment is effective for subjects carrying a particular mutation, not effective for subjects carrying a particular mutation, etc. Such querying can be carried out prospectively or retrospectively on the database by any suitable means, but is generally done by statistical analysis in accordance with known techniques, as described herein.

Another embodiment of the present invention is a method of treating a subject with DMXL-associated mental retardation, comprising the step of in vivo introduction into said subject a therapeutically effective amount of a nucleic acid (e.g., a heterologous or exogenous nucleic acid) encoding a DMXL1 protein and/or DMXL2 protein. An alternative method of the present invention is treating a subject with DMXL-associated mental retardation, comprising the steps of introducing ex vivo into a cell a therapeutically effective amount of a nucleic acid (e.g., a heterologous or exogenous nucleic acid) encoding a DMXL1 protein and/or DMXL2 protein and introducing said transformed cell into said subject. In some embodiments, said introduction also includes introduction of a suicide gene. Such gene therapy methods are well known in the art and one of ordinary skill would be able to select from a wide variety of gene therapy vectors and protocols for delivering a nucleic acid encoding a DMXL1 protein and/or DMXL2 protein to a subject in need thereof and target the delivery of the nucleic acid appropriately to achieve the desired therapeutic effect.

The terms “exogenous” and/or “heterologous” as used herein can include a nucleic acid molecule or nucleotide sequence that is not naturally occurring in the nucleic acid construct and/or delivery vector (e.g., virus delivery vector) in which it is contained and can also include a nucleotide sequence that is placed into a non-naturally occurring environment and/or position relative to other nucleotide sequences (e.g., by association with a promoter or coding sequence with which it is not naturally associated). A heterologous or exogenous nucleotide sequence or amino acid sequence of this invention can be any heterologous nucleotide sequence and/or amino acid sequence that has been introduced into a cell and can include a nucleotide sequence and/or amino acid sequence for which an original version is already present in the cell and the heterologous nucleotide sequence and/or amino acid sequence is a duplicate of the original naturally occurring version, and/or the heterologous nucleotide sequence or amino acid sequence can be introduced into a cell that does not naturally comprise the same nucleotide sequence and/or amino acid sequence.

The nucleic acid of this invention can be present in a vector and such a vector can be present in a cell. Any suitable vector is encompassed in the embodiments of this invention, including, but not limited to, nonviral vectors (e.g., plasmids, poloxymers and liposomes), viral vectors and synthetic biological nanoparticles (BNP) (e.g., synthetically designed from different adeno-associated viruses, as well as other parvoviruses).

It will be apparent to those skilled in the art that any suitable vector can be used to deliver a heterologous nucleic acid of this invention. The choice of delivery vector can be made based on a number of factors known in the art, including age and species of the target host, in vitro vs. in vivo delivery, level and persistence of expression desired, intended purpose (e.g., for therapy or polypeptide production), the target cell or organ, route of delivery, size of the isolated nucleic acid, safety concerns, and the like.

Suitable vectors also include virus vectors (e.g., retrovirus, alphavirus; vaccinia virus; adenovirus, adeno-associated virus, or herpesvirus), lipid vectors, poly-lysine vectors, synthetic polyamino polymer vectors that are used with nucleic acid molecules, such as plasmids, and the like.

Any viral vector can be used in the present invention. Examples of such viral vectors include, but are not limited to, vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Geminivirus; Group Giardiavirus; Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picornaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and Plant virus satellites. Protocols for producing recombinant viral vectors and for using viral vectors for nucleic acid delivery can be found, e.g., in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Nonlimiting examples of vectors employed in the methods of this invention include any nucleotide construct used to deliver nucleic acid into cells, e.g., a plasmid, a nonviral vector or a viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). For example, the recombinant retrovirus can then be used to infect and thereby deliver a nucleic acid of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Also included are chimeric viral particles, which are well known in the art and which can comprise viral proteins and/or nucleic acids from two or more different viruses in any combination to produce a functional viral vector. Chimeric viral particles of this invention can also comprise amino acid and/or nucleotide sequence of non-viral origin (e.g., to facilitate targeting of vectors to specific cells or tissues and/or to induce a specific immune response). The present invention also provides “targeted” virus particles (e.g., a parvovirus vector comprising a parvovirus capsid and a recombinant AAV genome, wherein an exogenous targeting sequence has been inserted or substituted into the parvovirus capsid).

Physical transduction techniques can also be used, such as liposome delivery and receptor-mediated and other endocytosis mechanisms (see, for example, Schwartzenberger et al., Blood 87:472-478, 1996). This invention can be used in conjunction with any of these and/or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468, (1990); and Wolff, Nature 352:815-818, (1991).

Thus, administration of the nucleic acid of this invention can be achieved by any one of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the methods described herein. Furthermore, these methods can be used to target certain diseases and tissues, organs and/or cell types and/or populations by using the targeting characteristics of the carrier, which would be well known to the skilled artisan. It would also be well understood that cell and tissue specific promoters can be employed in the nucleic acids of this invention to target specific tissues and cells and/or to treat specific diseases and disorders.

An effective amount of a composition of this invention will vary from composition to composition and subject to subject, and will depend upon a variety of factors such as age, species, gender, weight, overall condition of the subject and the particular disease or disorder to be treated. An effective amount can be determined in accordance with routine pharmacological procedures known to those of ordinary skill in the art. In some embodiments, a dose ranging from about 0.1 μg/kg to about 1 gm/kg will have therapeutic efficacy. In embodiments employing viral vectors for delivery of the nucleic acid of this invention, viral doses can be measured to include a particular number of virus particles or plaque forming units (pfu) or infectious particles, depending on the virus employed. For example, in some embodiments, particular unit doses can include about 10³, 10⁴, 10⁵, 10⁶, 10⁷, 10⁸, 10⁹, 10¹⁰, 10¹¹, 10¹², 10¹³ or 10¹⁴ pfu or infectious particles.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year and/or as necessary to control a particular condition and/or to achieve a particular effect and/or benefit. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

The present invention is more particularly described in the following examples that are intended as illustrative only since numerous modifications and variations therein will be apparent to those skilled in the art.

### EXAMPLES

### Example I

### Studies on DMXL1 and DMXL2 Mutations

Initial observation of a mentally retarded familial adenomatous polyposis (FAP) patient with deletion of the APC gene associated with FAP. A 5-year-old child was referred to the DNA diagnostic laboratory for mutation analysis of the APC gene associated with FAP. Full sequencing of the coding region of the APC gene did not identify any disease-causing mutation. Further analysis was performed to detect gross rearrangements in the APC gene by real-time PCR. This analysis showed the deletion of one copy of the APC gene. Subsequent fluorescence in situ hybridization (FISH) analysis identified the breakpoints of this deletion as 5q22-5q23.2. This patient was reported to be mentally retarded and had additional clinical features, such as obesity, hypotonia, hoarse cry, high prominent forehead, flat temporal regions, and thin and arched eyebrows. Subsequently, two additional patients aged 3 and 10 years were identified with a complete deletion of one copy of the APC gene that also encompassed the DMXL1 gene. The clinical features of these two patients were similar to the first identified patient.

To date, about 70 genes have been mapped within the deleted region, with the APC gene being the most clinically relevant gene at 5q22.2 [45, 46]. The MCC gene, at 5q22.2, also plays a role in colorectal cancer [47]. The Dmx-like 1 (DMXL1) gene at 5q23.1 is expressed in a variety of tissues and seems to have an important regulatory function [48]. A “de novo” interstitial deletion 5q22q23.2 in a 34-year-old woman exhibiting schizophrenia has been described by Bennett et al. [49]. The patient was also moderately mentally retarded and showed discrete dysmorphic features, such as delays in speech and motor development, low-set ears, low posterior hairline, and tapered fingers. Another case described by Raedle et al. showed some characteristics similar to those of the patients described herein [50]. The patient described by Raedle et al. was not affected by schizophrenia or any other major psychosis. Garcia-Minaur et al. concluded that there is a general phenotype for proximal deletions that encompasses the 5q22.3q23.2 region [51]. Individuals with distal deletions encompassing the q22-q31 region also share a consistent phenotype. Herrera et al. and Hockey et al. also described mentally retarded individuals with 5q deletions who also exhibited multiple developmental abnormalities and FAP. These data suggest that genes responsible for normal mental function are included in the region [52].

DMXL1 gene. The DmX gene was isolated from the X chromosome of Drosophila melanogaster. TBLASTN searches of the dbEST databases revealed sequences with a high level of similarity to DmX in a variety of different species, including insects, nematodes, and mammals, which shows that DmX is an evolutionarily highly conserved gene [48]. The human ortholog of DmX, Dmx-like 1 (DMXL1), is localized to 5q23.1 with a genomic size of ˜176 kb. The human DMXL1 gene contains 43 exons and codes for a large mRNA of 11 kb with an open reading frame of 3027 amino acids. The putative protein belongs to the superfamily of WD-repeat proteins, which have mostly regulatory functions. The DMXL1 protein contains an exceptionally large number of WD-repeat units. The DMXL1 gene is located on chromosome 5q23.1 as determined by radiation hybrid mapping and FISH. The DMXL1 gene was recently reported to be down regulated in astrocytomas, though the significance of this down regulation is unknown [53].

DMXL1 gene and its paralogous gene DMXL2. RAB-3 is a 12 WD-domain protein, which binds both GDP/GTP exchange protein and GTPase-activating protein [54-56]. These domains are found in a variety of proteins and are likely to be involved in protein-protein interactions. RAB3 shows a domain structure similar to that of DMXL1, which has 10 WD domains, and has been renamed DMXL2 [56]. Phylogenetic analysis confirms that both genes are paralogs, with invertebrate sequences basal to vertebrate DMXL1 and DMXL2. Thus, the duplication event that gave rise to DMXL1 and DMXL2 most likely involved two rounds of “en bloc” duplication in early vertebrate ancestry [57].

The DMXL2 gene at 15q21.2 is reported to be deleted in 15q21 syndrome [58-62]. Interstitial deletions of chromosome 15q not involving the PWS/AS region are uncommon and poorly characterized [59, 63]. Few cases defined at the cytogenetic level have been reported with 15q21 deletions, and characteristic facial dysmorphisms are present in all of them. The DMXL2 gene is localized to 15q21.2 with a genomic size of ˜163 kb. The human DMXL2 gene contains 43 exons and codes for a large mRNA of 9 kb with an open reading frame of 3036 amino acids. DMXL1 and DMXL2 are 54% identical and 69% similar at the amino acid level and contain WD repeats. The DMXL1 and DMXL2 proteins have not been crystallized, therefore the structure of these proteins is not known.

Based on the clinical presentation of the patients described herein and of patients described in the literature, mutation analysis of the DMXL1 and DMXL2 genes was carried out for patients who had a PWS-like presentation and had previously tested negative for the methylation assay. Prader-Willi syndrome (PWS) is a developmental disorder characterized by mental retardation or learning disability, infantile hypotonia and poor suck reflex, growth retardation, and childhood onset of pronounced hyperphagia. Motor milestones and language development are delayed. All individuals have some degree of cognitive impairment (Table 1). A distinctive behavioral phenotype (with temper tantrums, stubbornness, manipulative behavior, and obsessive-compulsive characteristics) is common. PWS is caused by the absence of the paternally derived PWS/AS region of chromosome 15 via one of several genetic mechanisms.

A total of 114 samples of previously extracted DNA samples were obtained from the Greenwood Genetics Center, NC and the Emory Genetics Laboratory (EGL) and were screened for mutations in the DMXL1 and DMXL2 genes. To date, bidirectional sequencing has been completed of all samples for DMXL1 gene and for 25 samples for mutations in the DMXL2 gene. The DMXL1 and DMXL2 genes are large genes containing 43 exons each. All exons of the DMXL1 gene and the 100-bp flanking intronic region were amplified at a universal PCR cycle and sequenced bidirectionally. Sequences were assembled with a GenBank® database wild-type consensus sequence using Mutation Surveyor software. Eleven novel heterozygous missense variants were identified in the DMXL1 gene, and two novel heterozygous missense variants were identified in the DMXL2 gene (Table 2).

All mutations were confirmed by re amplifying the targeted exon and resequencing bidirectionally. Mutations were also confirmed by bidirectional sequencing with an alternate primer set. These variants were not found in the dbSNP database. Parental samples have been obtained for three cases and it has been demonstrated that the three missense mutations, R1826C, M1983V and R2056C, were de novo whereas common polymorphisms documented in the dbSNP database were found to be inherited. There is an ongoing process of obtaining parental samples from the remainder of cases to determine whether these missense mutations are inherited or occur de novo. Clinical assessment and follow-up contact of patients and their families for the remainder of cases is also ongoing.

The 13 variants found are predicted to be deleterious according to the SIFT (sorting tolerant from intolerant), POLYPHEN (polymorphism phenotyping), and PANTHER (protein analysis through evolutionary relationships) prediction programs. These missense variants were not detected in 1800 normal chromosomes. The controls analyzed included 1000 normal Caucasian samples and 800 samples from a mixed ethnic panel, including Chinese, Mexicans, and Asians. These data indicate that mutations in DMXL1 and DMXL2 genes are associated with a novel but previously undescribed syndrome with clinical features including mental retardation.

Deletion/duplication analysis: A targeted 4-plex 385K comparative genomic hybridization (CGH) array has been designed for detecting single and multiple exon deletions and duplications in DMXL1 and DMXL2 genes. The CGH array includes oligonucleotides for the DMXL1 and DMXL2 genes and several other loci that are known to be associated with a Prader-Willi-like phenotype (Table 3). The CGH array design is exon centric and has an average spacing of five base pairs in the targeted loci. Five wild-type samples have been analyzed on the array. The data for the wild-type samples are robust. No copy number variants (CNVs) were identified in the wild-type samples, but it is expected that CNVs will be identified as the data set gets larger. The CNVs will be compared with the previously documented CNVs in the UCSC database. CGH array analysis will be performed on all DNA samples from the Greenwood Genetics Center, NC and the EGL. This analysis will be performed regardless of the findings from the sequence analysis of the DMXL1 and DMXL2 genes. Performing sequence analysis for the DMXL2 gene and large deletion/duplication analyses for DMXL1 and DMXL2 genes will make the mutation analysis comprehensive. Despite the maturity of sequencing and CGH, an estimated 1-2% of mutations located deep within intronic or promoter regions cannot be identified. An initial study of 15 samples employing CGH showed no deletion.

Allele-specific methylation analysis. To determine the methylation status of the DMXL1 and DMXL2 gene promoters, a real-time assay was designed. Two allele-specific primer/probe sets have been designed; the first set is labeled with FAM and targeted to the methylated copy, while the second is labeled with HEX and targeted to the unmethylated copy. Real time methylation PCR analysis showed that both the DMXL1 and DMXL2 genes are not imprinted. Another set of primers targeted to exon 5 of the DMXL1 gene and exon 11 of the DMXL2 gene with VIC and NED label was designed to use as an internal gene control. The chromosome 5q22 U2AFBPL gene, which is proximal to the DMXL1 gene and is known to not be imprinted in humans, was an independent assay control. Gain of methylation at the H19 locus on chromosome 11p15.5 implicated in Beckwith-Wiedemann syndrome (BWS) was used as a methylation control.

Twenty wild-type samples have been analyzed to determine the efficacy of bisulphite treatment and validate the real-time PCR methodology for the U2AFBPL gene and H19 locus. Real time PCR performed on the promoter regions of the DMXL1 and DMXL2 genes showed that these two genes are not imprinted.

No nonsense or truncating mutations in the DMXL1 or DMXL2 genes have yet been identified since the identification of the original patient with deletion of 5q22-5q23.2 encompassing the APC and DMXL1 genes. The results from the FAP patient with MR and other patients described in the literature indicate that heterozygous deletion of the DMXL1 gene is not lethal in humans. Mutation analysis in additional patients may identify nonsense or truncating mutations.

Yeast ortholog RAV1. Rav1 is a part of the RAVE complex that comprises Rav1, Rav2 and Skp1. Rav1 is important in forming a functional V-ATPase on vacuolar and early endosome membrane. Vacuolar type H+-ATPase (V-ATPase) is a highly evolutionarily conserved enzyme with remarkably diverse functions in eukaryotic organisms[64]. V-ATPases acidify a wide array of intracellular organelles and pump protons across the plasma membranes of numerous cell types. V-ATPases couple the energy of ATP hydrolysis to proton transport across intracellular and plasma membranes of eukaryotic cells. In vivo regulation of V-ATPase activity is accomplished by reversible dissociation of the Vₗ domain from the Vₒ domain. After initial assembly, the yeast V-ATPases can reversibly disassemble into free Vₒ and Vₗ domains after 2-5 min of glucose deprivation. Reversible disassembly may be a general mechanism of V-ATPase activity regulation, since it exists in yeast.

Reassembly is proposed to be aided by a complex termed RAVE (regulator of H+-ATPase of vacuolar and endosomal membranes). It has been shown that Rav1p is essential for assembly and activation of the proton-translocating vacuolar ATPase (V-ATPase) both at the yeast lysosome (or vacuole) and at early endosomes [65-67]. The likely Saccharomyces cerevisiae (budding yeast) putative ortholog of DMXL1, Rav1p, has been identified through the isolation of suppressors of a mislocalized mutant form of the trans Golgi network (TGN) membrane protein, Kex2 protease [68-71]. The role of Rav1p in early endosome-to-PVC transport has been demonstrated and some interacting proteins, such as Skp1, have been identified. These studies in yeast provide a compelling model of the function of DMXL proteins.

### V-ATPase and Human Diseases.

Osteopetrosis. Osteopetrosis is generic name that represents a group of heritable conditions in which there is a defect in osteoclastic bone resorption. Both dominant and recessive osteopetrosis occur in humans [72,73]. Autosomal dominant osteopetrosis shows mild symptoms in adults who experience frequent bone fractures due to brittle bones. A form of osteopetrosis that is clinically more severe is termed autosomal recessive infantile malignant osteopetrosis [74,75]. Three genes have been identified that are responsible for recessive osteopetrosis in humans. Interestingly, they are all directly involved in the proton generation and secretion pathways that are essential for bone resorption. One gene is carbonic anhydrase II (CAII) that when mutated causes osteopetrosis with renal tubular acidosis (type 3) [76]. Mutations to the chloride channel CIC7 gene also lead to both dominant and recessive osteopetrosis [72].

Distal renal tubular acidosis (dRTA). The importance of V-ATPase activity in renal proton secretion is highlighted by the inherited disease distal renal tubular acidosis. Twelve different mutations to V-ATPase isoform B1 [77] and twenty-four different mutations in a4 lead to dRTA [78, 79].

Functional analysis of DMXL1 and DMXL2 gene mutations: RAV1 has been shown to be the putative yeast ortholog of the human DMXL1 gene [71]. Rav1p is a subunit of the RAVE complex (Rav1p, Rav2p, Skp1p), which promotes assembly of the V-ATPase holoenzyme. It is required for transport between the early and late endosome/PVC and for localization of trans-Golgi network (TGN) membrane proteins[71]. It is also a potential Cdc28p substrate. The rav1 Δ phenotype is viable, exhibits growth defects on a non-fermentable (respiratory) carbon source, and has been shown to be glycerol, Zn²⁺, and Co²⁺ hypersensitive. It accumulates early endosome-like structures and is known to decrease the rate of trafficking from an early endocytic intermediate to the vacuole. Rav1p has also been shown to function at the early endosome to regulate endocytic trafficking to the vacuole and localization of trans-Golgi network transmembrane proteins. The yeast model system, Saccharomyces cerevesiae will be used to perform functional analyses of DMXL1 and DMXL2 mutations.

Analysis of rav1Δ phenotypes to study the Rav1p mutations in budding yeast Saccharomyces cerevisiae. Rav1⁻ associated phenotypes have been extensively studied and the complete phenotype listing is available in the Saccharomyces genome database (SGD). Zinc hypersensitivity has been confirmed, as well as the decrease in respiratory growth rate on glycerol associated with rav1Δ [80] using a rav1Δ mutant made in Dr Robert Fuller's lab in W303 background (MATα ade2-1 can1-100 his3-11,15 leu2-3,112 trp1-1 ura3-1).

In vivo analysis of three non-synonymous changes modeled in budding yeast Saccharomyces cerevisiae to assess the functional effect of DMXL1 human mutations. The effect of three mutations was analyzed—two missense and one in frame deletion in the budding yeast, Saccharomyces cerevesiae. The three Rav1p mutants—Rav1pM998I corresponding to M1664I in DMXL1 (humans); Rav1pR1323C corresponding to R2056C in DMXL1 (humans), and 1014 ΔAAL corresponding to 1680 in DMXL1 (humans) were generated by site-directed mutagenesis using oligonucleotides (Integrated DNA Technologies) encoding the M998I and R1323C amino acid (aa) substitution, and the synthetic in frame deletion of three amino acids AAL at 1014 using a p413ADHSOI3 plasmid template encoding SOI3/RAV1 with a QuikChange Site-directed Mutagenesis Kit (Stratagene). The three amino acid (aa) in frame deletion is from a seven aa conserved region, AALKNAF, between Rav1p, DMXL1 and DMXL2. The in frame deletion was created to understand the effect of deletion of amino acid in the region that is conserved and similar in both humans and yeast. The plasmid template is a single copy centromeric plasmid in which RAV1 is expressed under a moderate ADH promoter. All constructs were sequenced bidirectionally to ensure the presence of each desired mutation and the absence of any additional mutations.

Rav1p mutations demonstrate glycerol sensitivity in wild type (WT) and rav1Δ cells. WT and mutant transformants selected on a synthetic complete (SC) medium lacking histidine and including 2% glucose (-his/Glu) were streaked onto -his/Glu again (as a control) and -his/Gly. All transformants grew to a similar extent on glucose synthetic medium. On medium containing glycerol, rav1 deletion transformants expressing the missense alleles could partially compensate for wild-type Rav1p while the in frame deletion of AAL abrogates Rav1p's ability to restore growth on glycerol containing media. Quantitative growth curve analysis is pending. Growth of WT transformants was also checked as a control on glycerol medium. As reported by Fuller [81], transformants expressing RAV1 do not grow; over expression of RAV1 is toxic to cells. The R1323C RAV1 allele demonstrated the least sensitivity to glycerol, while the M998I RAV1 allele increased glycerol sensitivity.

Rav1p mutations demonstrate Zn⁺⁺ hypersensitivity in WT and rav1Δ cells. WT and mutant transformants selected on a synthetic complete (SC) medium lacking histidine and including 2% glucose (-his/Glu) were streaked onto yeast adenine peptone dextrose (YAPD) containing 5 mM ZnCl₂ and without zinc as a control. This medium was buffered with 50 mM Na-MOPS, pH 5.6 and 50 mM Na-MES, pH 5.6 as the rav1Δ mutant used in this study is pH sensitive. Since the rav1Δ strain is Zn⁺⁺ hypersensitive, selection pressure is not required for growth. All transformants grew well on medium lacking zinc. On medium containing zinc, transformants expressing the missense alleles had a moderate effect on growth while the in frame deletion of AAL completely abrogates Rav1p's ability to restore growth on zinc containing media. Quantitative growth curve analysis confirmed the plate data. Growth curve analysis of WT transformants was also done as a control and all grew to the same extent in medium containing zinc. A Rav1 plasmid with HA epitope will be used for site directed mutagenesis in order to perform protein expression studies to check if comparable levels of proteins are made as compared to WT Rav1p.

Rav1p mutations disrupt endocytosis in WT and rav1Δ cells. The internalization of the lipophilic dye FM4-64 was monitored to determine if there were defects in endosome to vacuole trafficking upon reduction of function of Rav1. FM4-64 incorporates into the plasma membrane and traffics to the vacuole via early and late endosomes [82]. In wild type cells, FM4-64 is transported to the vacuole in a matter of minutes at 30° C., while in cells with endocytic defects, FM4-64 accumulates in punctate spots in the cytoplasm and is unable to transit out of these punctate regions.

To assess the effects of loss of Rav1p function on endocytosis, WT and rav1Δ cells were grown overnight in YPD to an OD₆₀₀ of 0.9. Ten ml cultures were harvested, and resuspended in 500 ul of cold -his/Glu+40 uM FM4-64 (Molecular Probes). Cells were incubated for 30 min on ice. The cells were subsequently washed to remove the dye and kept shaking at 30° C. for 30 mins. The cells were harvested, washed with 100 ul of 10 mM NaN₃, 1 mM NaF, and resuspended in 2 5 ul of 10 mM NaN3, 10 mM NaF. FM4-64 staining was seen using an Olympus BX60 epifluorescence microscope equipped with a Photometrics Quantix digital camera. Extensive vacuolar staining was observed in WT cells after 30 min at 30° C., while in rav1Δ cells, 50% of cells showed vacuolar ring like structures and 50% of cells showed punctate like structures, suggesting a decreased rate of trafficking from an early endocytic intermediate to the vacuole.

Wt and rav1Δ transformants were harvested from -his/Glu in a manner similar to that stated above. All the mutant alleles altered the vacuolar staining as compared to WT allele or just empty vector. Extent and severity of defect varies between the different mutants. The effect was more pronounced in WT cells than in rav1Δ cells. The rav1Δ cells show decreased rate of trafficking from an early endocytic intermediate to the vacuole [81] and subsequently some percentage of cells show vacuolar staining and some show punctate like structures. Expression of WT RAV1 almost completely restores vacuolar staining while missense alleles show a mixed population of cells with normal and abnormal endocytosis. The deletion allele shows more percentage of cells with abnormal endocytosis. More quantitative assessment will be needed to document the differences in rav1Δ transformants.

Yeast two-hybrid screen. DMXL1, DMXL2 and Rav1p are expected to possess several WD-40 repeats that likely mediate interactions with other proteins. Therefore it is critical to identify the proteins with which DMXL1 and DMX12 interact.

Bait construct. A 2 kb region of DMXL1 has been cloned, showing highest homology to RAV1 in frame with the Gal4 DNA-binding domain of pGBKT7. cDNA was made with the Roche Transcriptor First Strand cDNA Synthesis Kit using random hexamer primer from RNA isolated from human muscle tissue. cDNA was used to PCR amplify the portion of DNA to be cloned using primers with EcoRI and BamH1 restriction sites into pGBKT7 using Phusion High-Fidelity DNA Polymerase. The construct has been checked by restriction enzyme digestion and bidirectional sequencing. The bait did not display any toxicity towards yeast cells, does not auto-activate and is expressed in yeast.

Bait construct checked for autoactivation. Bait protein was expressed as a fusion with the Gal4 DNA-BD in yeast strain AH109. The high-complexity pretransformed cDNA library, which expresses fusions with the Gal4 AD, is provided in yeast strain Y187. When cultures of the two transformed strains are mixed together overnight, they mate to create diploids. Diploid cells contain four reporter genes: HIS3, ADE2, MEL1, and LacZ, which are activated in response to two-hybrid interactions. To test whether bait autoactivates, the transformation of the bait and pGBKT7-p53 (vector) that serves as a negative control was carried out, using the lithium acetate method, into AH109 cells. AH109 cells expressing vector, bait, ISO4NT were mated with Y187 yeast cells expressing pACT2, SEC3 on YPD. The AH109 and Y187 transformants were selected on -trp and -leu media, respectively. When bait and library (prey) fusion proteins interact, the DNA-BD and AD are brought into proximity to activate transcription of reporter genes such as ADE2, HIS3. The SEC3-ISO4NT interaction serves as a positive control. The YPD plate was replica-plated onto -leu-trp to select for diploids. The diploids were then streaked out on -leu-trp (-L-T) again and on -leu-trp-his-ade to check for autoactivation. The -L-T plate was replica plated onto -leu-trp-his-ade showing the same results. The streaked plates were checked after 5 days and 8 days. Autoactivation is not seen, as no growth is seen on pACT2 X bait construct.

Bait construct checked for expression in yeast. A Western blot was performed to check if the bait is expressed well in yeast. AH109 cells transformed with pGBKT7-p53 vector that is tagged with myc epitope served as a positive control and bait constructs were grown overnight in 10 ml -trp/Glu.

Proteins were separated using a pre-cast Bio-Rad 4-20% gradient gel and protein was detected by Western blot using a mouse monoclonal antibody against Myc. The expression of bait construct is weak and the size is as expected; around 100 kDa. The smaller ˜35 kDa product could be a degradation product.

The DMXL1 and DMXL2 genes will be examined for point mutations, large deletions/duplications, and sequence variations in 200 patients and their parents presenting with PWS-like phenotypes but without 15q11-q13 involvement. Missense mutations in the DMXL1 and DMXL2 genes are dominant mutations and cause a Prader Willi syndrome (PWS) like phenotype. The goal of these studies is to characterize the phenotype caused by mutations in the DMXL1 and DMXL2 genes, which will give information about the genotype:phenotype correlation.

Fourteen novel missense mutations in the DMXL1 gene on chromosome 5q23.1 in 12.3% (14/114) and two novel missense variants in the DMXL2 gene in 8% (2/25) of patients with a PWS-like phenotype who previously tested negative for known chromosome 15 etiologies have now been identified. Analysis of parental samples previously submitted to the laboratory along with the probands sample from three cases showed these missense mutations (R1826C, M1983V, R2056C) are all de novo whereas the common polymorphisms were found to be inherited.

EGL has a repository of samples that have been collected over the past few years referred to the laboratory for PWS testing. These samples are available for mutation detection in DMXL1 and DMXL2 genes. Samples are stored as extracted DNA, which has been quantified and checked for quality. The clinical phenotype for each sample is available in the individual databases. A total of 200 samples will be obtained from the EGL repository of patients who have a PWS-like phenotype or closely resemble the phenotype of the patients in whom mutations have been found. The reported clinical features in these patients include: mental retardation, hypotonia, failure to thrive, late-onset obesity, low-set, dysplastic ears, down turned corners of the mouth, and small hands and feet.

Clinical assessment of patients and refinement of clinical phenotype associated with mutations in the DMXL1 and DMXL2 genes: A clinical assessment form has been developed that will be used to collect the phenotype information for patients in these studies. Each patient will be contacted and brought to the EGL clinic, where the patient will be evaluated clinically. Data collected on the clinical assessment form will be maintained in a database. The clinical phenotype of patients with DMXL1 and/or DMXL2 gene mutations will be defined by obtaining a complete medical and family history of the patient in whom a mutation is identified. The patients will be thoroughly evaluated for the previously reported features (Table 1). Any additional and clinically important features will be recorded and linked to the mutation database. These data will include all the points on the assessment sheet and also additional features as noted by the clinician. A detailed medical and family history will be obtained from these patients in order to characterize the novel syndrome associated with mutations in the DMXL1 and DMXL2 genes.

Parental samples will be obtained from patients in whom a mutation is found in order to determine whether the mutation is de novo or inherited. An extensive medical evaluation will be carried out of the parent of an offspring in whom a mutation is detected. Fibroblast cell lines from these patients are also expected to be established for future functional studies. All mutation data will be stored in the same database in order to examine the genotype:phenotype correlation. The patients will be followed for a period of five years through the EGL on a yearly basis.

### Mutation Detection.

PCR analysis. Patient DNA will be PCR amplified for the DMXL1 and DMXL2 genes using PCR primers designed to cover the entire coding region of the gene along with intronic sequence, upstream promoter, and downstream regulatory regions. All DMXL1 gene and DMXL2 gene exons will be amplified under common PCR conditions using the Roche Applied Science GC-Rich Kit for fragment 10 and FastStart Taq DNA Polymerase for all other fragments.

DNA sequencing analysis. The PCR products will be purified using the Roche PCR Purification Kit. Purified products will be sequenced using the ABI SoLiD High throughput sequencer and base caller according to the manufacturer's instructions.

Sequence analysis. Patient sequences will be aligned to wild-type sequence for DMXL1 and DMXL2 genes using the NextGENe™ and Mutation Surveyor™ alignment software (Softgenetics, Inc). NextGENe and Mutation Surveyor are sequence alignment software programs used for mutation detection and allow for base calling and they also report zygosity. Data for each patient for each gene will be saved and archived as individual projects. The nucleotide numbering will be reported using the sequence of GenBank® database Accession No. NM_{—}005509 for the DMXL1 gene (SEQ ID NO:1) and the sequence of GenBank® database Accession No. NM_{—}015263 for the DMXL2 gene (SEQ ID NO:3). Amino acid residue numbering is based on the amino acid sequence of DMXL1 (SEQ ID NO:2) and the amino acid sequence of DMXL2 (SEQ ID NO:4). A database of sequence variants and mutations found in the laboratory will be created. All variants will be color-coded for zygosity and their status. (Homozygous—Pink, Heterozygous—Blue; if the variant is a likely mutation, it will be underlined and highlighted yellow in the database). The mutation status will also be entered in the clinical data sheet for each patient.

Sequence nomenclature and classification: All sequence variants will be reported using standard sequence nomenclature as described by HUGO and the Human Genome Variation Society (HGVS). Sequence variants called by the Mutation Surveyor software will be checked manually by identifying the base position on the cDNA sequence for each gene. Previously reported single nucleotide polymorphisms (SNPs) will be checked in the dbSNP database. Any novel sequence variant, both silent and missense, will be analyzed for its effect on the DMXL1 and DMXL2 transcripts using several different analysis programs, all of which are free and open-access programs.

SIFT (Sorting intolerant from tolerant) predicts whether an amino acid substitution affects protein function based on sequence homology and the physical properties of amino acids. SIFT can be applied to naturally occurring nonsynonymous polymorphisms and laboratory-induced missense mutations. Given a protein sequence, SIFT will return predictions for what amino acid substitutions will affect protein function. SIFT is a multistep procedure that searches for and chooses similar sequences, makes an alignment of these sequences, and calculates scores based on the amino acids appearing at each position in the alignment.

POLYPHEN (Polymorphism phenotyping) is a tool that predicts the possible impact of an amino acid substitution on the structure and function of a human protein using straightforward physical and comparative considerations.

PANTHER (protein analysis through evolutionary relationships) classification system is a unique resource that classifies genes by their functions using published scientific experimental evidence and evolutionary relationships to predict function even in the absence of direct experimental evidence. Proteins are classified by expert biologists into families and subfamilies of shared function, which are then categorized by molecular function and biological process ontology terms. For an increasing number of proteins, detailed biochemical interactions in canonical pathways are captured and can be viewed interactively:

All the identified mutations will be entered into the DMXL1 and DMXL2 database in the laboratory. The database will link the clinical phenotype for each patient with the laboratory findings. All missense and silent variants will be analyzed further by targeted sequencing of 500 normal controls. If a mutation is identified in a sample from an ethnicity other than Caucasian, a targeted sequencing of 500 normal controls from that ethnicity will be performed. Targeted PCR amplification will be performed for reconfirming each novel variant and the controls sequenced bidirectionally. All novel variants will also be confirmed using an alternative primer set and by sequencing bidirectionally. Sequences will be analyzed using Mutation Surveyor software. Variants that occur at a frequency of ≧1% in the general population will be classified as polymorphisms and reported in the database accordingly.

Parental samples: Parental samples will be obtained from patients for whom a mutation has been identified. DNA from the parental blood sample will be extracted using a commercially available DNA extraction kit (Puregene, Qiagen INC). Targeted PCR amplification of the previously identified variant in the individual's offspring will be performed. The PCR products will be sequenced bidirectionally and assembled with the individual offspring's mutation surveyor project to identify the variant. If the variant is detected in the parent, it will be classified as a mutation or polymorphism after extensive clinical evaluation and comparison with data from the analysis of control samples. It is possible that some variants will not be classified at this stage and will require extensive functional analysis.

Genetic analysis may reveal that some of the mutations are inherited from a parent. A detailed analysis will be performed on the parent who carries the mutation for phenotypic features of the condition. If the parent does not show a phenotype it is possible that other modifying factors result in variable expressivity, which can compensate for the phenotypic effect in the parent. Discerning this type of interaction is not within the scope of these studies, although thorough documentation of the parent's entire clinical history may help in revealing such interactions.

Functional and biochemical analysis of the human mutations will be performed in a yeast model system Saccharomyces cerevesiae. Proteins that interact with DMXL1 and DMXL2 will be identified by yeast two-hybrid analysis.

Missense mutations in DMXL1 and DMXL2 genes, when introduced in the putative yeast ortholog RAV1, act as dominant mutations and disrupt the function of RAV1p by disrupting its binding with the interacting proteins such as Skp1 and the assembly and activation of the proton-translocating vacuolar ATPase (V-ATPase) both at the yeast lysosome (or vacuole) and at early endosomes.

Assessment of the effect of human mutations in a yeast model system by site-directed mutagenesis. RAV1 is 4.4 kb in size and 60% homologous to DMXL1 and 46% homologous to DMXL2 cDNA. The central part of DMXL1 and DMXL2 is the region with the highest level of similarity to the yeast Rav1. The yeast Rav1p codes for a putative protein of 1357 amino acids (aa) with a calculated mass of 155 kDa, which is significantly smaller than the DMXL proteins. However, the marked similarity between Rav1 and the central region of DMXL1/DMXL2 suggests that the central portion of DMXL1/DMXL2 and the RAV1 may share a common functional domain. Rav1p was identified through the isolation of suppressors of a mislocalized mutant form of the trans-Golgi network (TGN) membrane protein, Kex2 protease. It has been shown that Rav1p is essential for assembly and activation of the proton-translocating vacuolar ATPase (V-ATPase) both at the yeast lysosome (or vacuole) and at early endosomes.

In order to understand the interactions and functions of DMXL1 and DMXL2 in animal cells, studies will be conducted to examine the consequences of DMXL1 and DMXL2 mutations, when introduced at conserved sites in Rav1, on the function of the protein in yeast. To date, out of the 16 mutations identified in human patients in DMXL1 and DMXL2, 13 are completely conserved to Rav1. Four mutations out of 13 lie in the putative Rav1 functional domain encompassing amino acids (aa) 900 to 1206. Mutations found in human patients in the region of interest result in the following aa changes: E1623K, M16641, L1745R, and S1774N. They map to E951K, M998I, L1077R, and S1095N respectively, in Rav1p. Site-directed mutagenesis will be performed to obtain these rav1 mutant alleles using rav1 plasmids. They will be cloned into a suitable yeast expression vector. The mutations will be verified by bidirectional sequencing. Rav1 deletion strain will be transformed with the WT and mutant plasmids and empty plasmid to check their effects on phenotypes like glycerol sensitivity and Zn⁺⁺ hypersensitivity and endocytosis. Studies have already successfully shown that the expression of all three mutant alleles of RAV1—M998I, R1323C and ΔAAL in yeast have a dominant effect to varying degrees on the phenotypes tested. Studying the effect of these mutations on different phenotypes will allow for precise mapping of the functional domains of the DMXL1 and DMXL2 proteins. Additional mutations identified from analysis of patient samples will be also studied to understand their functional effect.

Assessment of the Rav1p mutations on endocytosis. The aberrant effect of Rav1p mutations on endocytic delivery to vacuole has been demonstrated by labeling with FM4-64. Further studies are being carried out in order to understand the effect of Rav1p mutation on endosomes and endocytic delivery. See, e.g., Table 5.

Assessment of the effect of Rav1p mutations on endosomes. In order to assess the effect of Rav1p mutations on endosomes, electron microscopic analysis will be performed, using thick sections (250 nm) analyzed at tilt angles to permit creation of stereo pairs [81, 97]. Sipos et al. [81] have previously shown that rav1 mutant cells massively accumulated ovoid membrane structures that consisted of clusters of basket-like structures formed from anastomosed tubules. Clusters occurred near the vacuole or facing finger-like invaginations of the plasma membrane, suggestive in appearance of early endosomal tubulo-vesicular structures shown to label with positively charged nanogold structures [98], which might be aberrant early or late endosomes, also were found adjacent to the vacuole, and, in some cases, seemed to be fusing with vacuoles.

For electron microscopy (EM), cells will be fixed, stained, and visualized [97]. For FM4-64 uptake experiments, strains GSY11-2A (soi3Δ) and GSY11-4D (SOI3) will be grown in YPD to ˜OD₆₀₀=0.9, harvested, and resuspended in cold YPD+40 μM FM4-64 (Molecular Probes). Cells will be then incubated on ice for 1 h, washed in cold YPD, and resuspended in 1 ml of cold YPD and incubated at 14° C., with shaking. After 40 min at 14° C., 500 μl of cells will be harvested and resuspended in 400 μl of prewarmed 30° C. YPD. Cells will be harvested, washed in 1×100 μl of 10 mM NaN₃, 0 mM NaF, resuspended in 25 μl of 10 mM NaN₃, 10 mM NaF, and stored on ice until they were examined using an Axioskop microscope (Carl Zeiss, Thornwood, N.Y.), recording data on film as described previously [99]. Rav1-GFP images will be examined using a Nikon Eclipse 800 microscope and an ORCA2 charge-coupled device (Hamamatsu, Bridgewater, N.J.). Cells will be grown in log phase in minimal medium at 25° C. and mounted in low-temperature agarose. Z-stacks will be created using eight 0.25-μm steps. ISEE Software (Inovision, Raleigh, N.C.) will be used for image capture and deconvolution of Z-stacks.

Assessment of the effect of rav1 mutant on endocytic delivery of FM4-64 to the vacuole. To assess the effects of loss of rav1p function on bulk-phase endocytosis, rav1 mutant cells will be stained, on ice, with the lipophilic fluorescence dye, FM4-64, warmed to 14° C. and 30° C. for 5 min, and then treated with sodium azide and sodium fluoride to deplete ATP. Previous work by Sipos et al. and these preliminary data suggest that at 14° C., FM4-64 accumulates in peripheral, punctate, endocytic structures in both wild-type and rav1 mutant cells, as has been observed previously for cells maintained at 15° C. [82]. When cells were shifted to 30° C. for 5 min, FM4-64 chased to the vacuolar membrane in wild-type cells but persisted in punctate structures in rav1 mutant cells. Extensive vacuolar membrane staining was observed in the rav1 mutant cells after 30 min at 30° C., although persistent staining of one or two extravacuolar punctate structures was still apparent. Internalization of FM4-64 from the plasma membrane to internal sites will be assessed by warming cells to 30° C., which will help determine the rate of trafficking from an early endocytic intermediate to the vacuole.

For colocalization of FM4-64 with Rav1-GFP, cells will be grown in log phase at 25° C. in 10 ml of YPD and then placed in a shaking water bath at 14° C. for 30 minutes prior to labeling with FM4-64. Cells will then be harvested, resuspended in 1 ml of YPD+40 mM FM4-64 pre-cooled to 14° C. and incubated at 14° C. for 30 minutes. Cells will be washed by filtration with YPD at 14° C. and placed on pre-cooled slides. A temperature-controlled stage (20/20 Technology, Inc. Wilmington, N.C.) will be used to maintain cells at 14° C. during visualization.

### Yeast Two Hybrid Analysis

Identification of proteins that interact with DMXL1 proteins using a yeast two-hybrid screen. Rav1p was originally isolated as a Skp1p-interacting protein that is part of a complex that contains Rav2p and the V1 subcomplex of the proton-translocating vacuolar ATPase (V-ATPase) [74,88]. Both RAV1 and RAV2 were shown to be important for glucose-regulated assembly of V1 onto V0 to form functional VATPase on the vacuolar membrane [74]. Analysis of the effects of Rav1p mutations demonstrates that loss of Rav1p function results both in alterations in the localization of trans Golgi network (TGN) membrane proteins as well as selective defects in delivery of endocytic cargo to the vacuole. A synthesis of these results suggests that assembly of vacuolar ATPase at the early endosome in yeast is essential for early endosome maturation and efficient transport from the early endosome to the prevacuolar compartment. RAV1p has been designated as a putative ortholog of DMXL1 based on the high degree of sequence homology in the putative functional domain.

Identification of proteins that interact with DMXL2 proteins using a yeast two-hybrid screen. A construct will be designed by cloning ˜1 kb of the DMXL2 coding sequence showing the highest homology with the putative yeast ortholog RAV1 to use as bait for the yeast two-hybrid experiments. This construct will be checked for appropriateness via bidirectional sequencing and restriction enzyme digestion. The bait will also be checked for any toxicity towards the yeast cells, auto-activation and expression in yeast in order to get it ready for the library screen.

Choice of library for yeast two-hybrid screen. DMXL1 and DMXL2 genes have been shown to be highly expressed in whole embryo and several tissues such as brain, cerebellum, skeletal muscle, bone marrow, spleen, pancreas, and heart (SAGE and GENENOTE dataset). Based on this, the matchmaker 11d mEmbryo library has been chosen for the yeast two hybrid experiments. The standard protocol by Clontech will be followed. At least one million diploids will be screened; using fewer would result in less chance of detecting genuine interactions. Phenotype will be confirmed by restreaking, yeast colony PCR, and rescue and isolation of the library plasmid responsible for activation of reporters. The interactions will be confirmed by co-immunoprecipitation and in vitro pull down assays. Similar experiments will be performed for DMXL2. The confirmed interactions will be used to study the effect of DMXL1 and DMXL2 gene mutations and for mapping the functional domains of the DMXL1 and DMXL2 genes.

Functional and behavioral analyses in a mouse model of both DMXL1 and DMXL2 genes. Based on the human phenotype, which encompasses a cytogenetic deletion involving DMXL1 and DMXL2 genes, heterozygous knockout models of Dmxl1 and Dmxl2 genes in mouse will be expected to have a similar phenotype and display behavioral changes.

In order to understand the biological function of DMXL1 and DMXL2 proteins, a mouse model for these two genes is employed, using gene-trapped ES cell lines. Mouse models have been previously used to understand gene function and pathways in diseases like hemochromatosis in which only missense mutations have been reported [83].

Mouse models of both DMXL1 and DMXL2 genes are produced using gene-trapped ES cell lines to study the biological function and behavioral effects. The effects of disrupting each of these two genes are analyzed in heterozygous and homozygous mice. By conducting behavioral assessment, the mouse phenotype can be compared with the human phenotype, to further define the novel syndrome.

Two ES cell lines have been selected, which are expected to result in altered/truncated protein without any residual activity. These cell lines were selected after carefully evaluating the alternative start sites or alternative splice sites of the DMXL1 and DMXL2 genes. The two cell lines have been provided by Bay Genomics (DMXL1; cell line id CSH215) and the Sanger Center, UK (DMXL2; AN0585). Both the ES cell lines have been verified by PCR amplification and Southern blot by these two providers. Blastocyst injections to generate chimeric mice and subsequent breeding to generate F1 progeny have been performed by the Emory University School of Medicine Transgenic Mouse and Gene Targeting Core Facility.

Dmxl1 gene. Germ-line transmission for Dmxl1 has been achieved: cell line id CSH215 targeted to exon 9/intron 9 junction of the Dmxl1 gene. Out of the 30 F1 progeny obtained, a total of 10 pups were heterozygous mutants for the Dmxl1 ES cell lines (FIG. 13). Germ-line transmission was confirmed by PCR-based analysis and walking of the targeted region carried out by PCR to identify the precise insertion point of the β-gal-neo-cassette. The β-gal-neo-cassette was found to be inserted 1 kb downstream of the exon9/intron9 junction. Real-time PCR studies of the DMXL1 cDNA confirmed heterozygous knockout of the Dmxl1 gene.

F2 progeny have been obtained from +/−(heterozygous males)×+/−(heterozygous females) breeding pairs. A total of 93 offspring were obtained. The expected frequency of genotypes if the homozygous mice survived according to Mendelian segregation is 47 heterozygotes, 23 wild type and 23 homozygous offspring. The observed frequency of the genotype is 54 heterozygotes and 39 wild type offspring. No homozygous offspring were obtained. All the offspring were analyzed by targeted PCR analysis to detect heterozygous insertion of the β-gal-neo-cassette. Analysis performed using Chi square test of Mendelian segregation indicates significance at a 1% level that the homozygous mutant animals die before weaning.

The time of death of the homozygous mutants during embryonic development is also being assessed. This is performed by picking several time points in gestation at which the embryos will be genotyped and morphological assessment of the embryonic and extraembryonic structures will be done. Phenotypic and behavioral analysis of the heterozygous offspring will also be carried out. A causal preliminary observation shows that the heterozygous offspring are sluggish in comparison to the wild type offspring.

Dmxl2 gene. Eight chimeras from ES cell line AN0585 have been obtained for Dmxl2, and germ-line transmission will be determined in the F1 progeny. The β-gal-neo-cassette is expected to insert 500 by from the exon 12/intron 12 junction.

Functional and behavioral assessment. Functional and behavioral assessment of mouse models of the DMXL1 and DMXL2 genes will be carried out. Expression analysis will be performed by in situ hybridization of the DMXL1 and DMXL2 genes in the brain during development, as this may indicate when the gene expression is important. If no live-born homozygous mice are obtained, morphological analysis of mouse embryos will be performed to look for morphological defects and associate them with the molecular defect, if possible.

Expression analysis of the DMXL1 and DMXL2 genes in adult wild-type and mutant mouse tissues. Quantitative real-time PCR analysis will be performed on different adult mouse tissues to reveal the expression patterns of the DMXL1 and DMXL2 genes.

DMXL1 and DMXL2 genes have been shown to be highly expressed in whole embryo and in several tissues such as brain, cerebellum, skeletal muscle, bone marrow, spleen, pancreas, and heart (SAGE and GENENOTE dataset). In order to understand tissue-specific expression of DMXL1 and DMXL2, real-time PCR analysis will be performed by extracting RNA from the adrenal gland, brain, cerebellum, heart, intestine, skeletal muscle, and spleen. Primers have been designed for two targeted exons in each gene to evaluate gene expression. The mRNA abundance of each transcript will be calculated relative to the expression of the housekeeping gene GAPDH (glyceraldehyde-3-phosphate-dehydrogenase). Data will be analyzed using the ABI analysis program, which analyzes the expression of a target gene relative to a nonregulated reference gene. The mathematical model used is based on the correction for exact PCR efficiencies and the mean crossing point deviation between sample group(s) and control group(s).

Behavioral assessment. All experiments will be performed in one of the two mouse behavioral testing rooms in the Whitehead Building vivarium. Naïve mice between 3 and 6 months of age are used for all experiments, unless use of younger or older mice is warranted. Both male and female mice are used for all experiments to test for sex differences; if none are observed, results will be combined. Mice will be acclimated to the behavior room for at least one week prior to experiments, and experiments using different paradigms will be separated by at least one week. Very stressful or potentially harmful tests (e.g., aggression, seizure susceptibility) will be performed last. Mice will be maintained on a 12-h light/dark cycle (lights on 0700, lights off 1900), and all tests will be conducted between 0900-1600, unless testing during the dark cycle is a necessary component of the paradigm. The number and types of paradigms examined can always be expanded to follow up on an initial finding or as a way to identify a potential phenotype if the initial tests fail to reveal one. The behavioral data will be analyzed by Student's two-tailed t-test when comparing two groups with equivalent variances, and Mann-Whitney U-test when comparing two groups with unequivalent variances. Multivariate analysis of variance will be used for comparison of three or more groups, and post-hoc comparisons will be performed using the Newman-Keuls test. An alpha level of 0.05 will be used for all statistical tests. The following is a description of the tests that will be performed.

A battery of tests has been chosen based on the clinical presentation of the patients in whom the novel missense mutations have been found. Mutant mice will be compared with appropriate controls in a battery of tests designed to assess spatial, social, and associative learning and memory, motor behavior, seizure susceptibility, anxiety, depression, and aggression. The number of tests and the order of testing will be tailored to each line of mice based on predictions of gene function. Based on the observed clinical phenotype in mice, additional behavioral assessments will be performed. It is possible that some additional features may be observed in mice that have not yet been seen in humans. The observations in mice will be correlated with the data with the human phenotype.

### Spatial Learning and Memory.

Morris water maze. The Morris water maze test is the canonical test for spatial memory in rodents and will be performed as described in the literature. The pool will consist of a steel circular water tank having an inner diameter of 115 cm and a height of 59.5 cm. The inner walls are painted white. The water level will be filled to 36.5 cm after being made opaque by the addition of four gallons of whole milk. Water temperature will be 31° C. initially, which typically declines to 26° C. by the end of the third training trial. The platform is white with a diameter of 10 cm, and the water level will be 1 cm above its top. It will be located half way from the center to the wall in the middle of one quadrant. Prominent spatial cues will be mounted on the walls or hung from the ceiling. A video camera attached to the Noldus EthoVision system will be mounted on the ceiling directly above the tank to monitor the activity of the mice and record the probe trials in an automated fashion. Mice will be placed in the pool between quadrants and the location will be varied randomly from trial to trial, except that the same location will never be used consecutively. Mice are allowed to search for the platform for 60 s prior to being removed from the pool. If the mouse climbs onto the platform prior to 60 s, it is allowed to remain on the platform for 10 s before being placed in a heated cage with dry paper towels. If the mouse jumps off the platform, the trial continues. The mice will be given three trials a day separated by about 2 h. The average escape latency on a given day will be calculated for each mouse. Swim speed will be determined by measuring the distance swum during each probe trial. The platform is removed for the probe trials, which last 60 s and do not begin until the mouse starts swimming (some mice occasionally float at the beginning of a trial). Measurements taken during the probe trial do not depend on swim speed.

### Social Memory.

Social recognition test. Social recognition will be assessed as described in the literature. All mice will be individually housed for at least one week prior to social memory testing. For the two days prior to testing, mice will be habituated to the stimulus animals (ovariectomized C57B16/J females). This is done to reduce the amount of sexual behavior exhibited by the males during testing. In trials 1-4, a stimulus animal (same animal for all four trials) will be placed in the male's home cage for 1 min with 10-min interatrial intervals. On the fifth trial, a novel stimulus animal will be placed in the male's cage for 1 min. Investigation time, which is defined as direct, active olfactory exploration of the stimulus female, will be scored from videotape. Sexual behavior, such as mounting, is not included in investigation time. Using this method, familiarity or social memory can be observed as a reduction in investigation time over the first four trials, with an increase back to baseline during the fifth trial when a novel animal is introduced.

Social discrimination test. The social discrimination test will be performed as described in the literature. In the first trial, a stimulus animal (ovariectomized female) will be placed into the male's home cage for 5 min and investigation time will be measured. Thirty minutes later, the male will be simultaneously exposed to the same stimulus animal from the first trial and a novel stimulus animal for 5 min. Social memory is assessed by comparing the amount of time the male spends investigating the familiar animal to the amount of time he spends investigating the unfamiliar stimulus animal. If the males recognize the previously presented female, they will spend more time investigating the novel female than the familiar female during trial 2. To demonstrate that any defects in this test are specifically “social” in nature, an object recognition test will also be performed. This test will be performed identically to the social recognition test, except that two novel objects (identical plastic balls) will be placed into the arena for 5 min instead of a female mouse, mice will be videotaped, and investigation time will be recorded. Thirty minutes later the animals will be put back into the same box where one familiar (one of the balls from trial 1) and one novel object (similar size ball, different in shape and color) will be placed. The behavior of mice will be videotaped, and time spent investigating each object will be recorded. If the animals recognize the previously presented ball, they will spend more time investigating the new ball than the familiar ball during trial 2.

### Associative Learning.

Place conditioning. Associative learning will be assessed by place conditioning as described in the literature. Mice are placed in the “neutral” middle compartment of a 3-compartment conditioned place preference chamber (San Diego Instruments, La Jolla, Calif.) and allowed to freely explore the other two compartments that are distinguishable by floor texture and wall pattern for 20 min, and time spent in each compartment is recorded (“pretest”). One to six days later, mice are subjected to “conditioning” sessions for three consecutive days. Mice are restricted to one compartment for 30 min in the morning, and restricted to the other compartment for 30 min in the afternoon (˜4 h after the morning conditioning session). One chamber is typically “neutral” (e.g., no associative stimulus), while the other chamber is typically paired with either a “rewarding” (e.g., food, cocaine injection) or “aversive” (e.g., lithium chloride injection) stimulus. Mice will be designated to receive the associative stimulus on either the “A” side or the “B” side using an unbiased design (i.e., for each treatment group, equal numbers of mice receive the associative stimulus on each side, and equal numbers of mice receive the associative stimulus on the “preferred” side and “non-preferred” side based on pretest results). The day following the last conditioning session, mice are placed in the neutral middle compartment in the absence of associative stimulus, allowed to freely explore all compartments for 20 min, and the time spent in each compartment is recorded. If the mouse associates the paired stimulus with positive feelings (reward), it will spend more time on the stimulus-paired side, while if the paired stimulus is aversive, the mouse will spend more time on the “neutral” (unpaired) side. The “preference” score is calculated by subtracting the amount of time spent on the neutral side from the amount of time spent on the stimulus-paired side.

### Depression.

Forced swim test. Depression will be assessed using a modified version of the Porsolt forced swim test. Mice will be forced to swim for 6 min in 20 cm of fresh 30° C. water in a 4-L beaker (18 cm diameter). In this test, struggling, swimming, and immobile floating behavior is typically observed, with immobility considered a “depression-like” phenotype. A mouse will be considered immobile when it is only making movements necessary to remain floating, and immobility will be quantified during the last 4 min of the test. The mice will then be removed from the water, dried, and returned to their home cage.

### Anxiety.

Open field test. The open field test will be performed as described in the literature. The open field apparatus is a circular arena (96.5 cm diameter) with opaque gray Plexiglas walls (28 cm high). A permanent marker is used to describe a smaller circle 18 cm from the walls that divide the chamber into a smaller inner circle (area=˜3100 cm²) and an outer ring (area=˜3800 cm²). Within the inner circle are 4 small PVC cylinders (3.5 cm high, 3.5 cm diameter opening) in a random arrangement that are used to measure investigatory behavior of the mice. Mice will be placed individually in the center of the inner circle and allowed to roam freely about the apparatus for 5 min. Measures will include (1) time spent in inner circle, (2) time spent in outer ring, (3) total number of crossings between the divisions, and (4) number of head-pokes into the cylinders. Mice that spend most of their time in the outer circle and against the walls are considered anxious, while mice that spend most of their time in the inner circle and exploring the cylinders are considered non-anxious.

Elevated plus maze. The elevated plus maze will be performed as described in the literature. The elevated plus maze consists of two open arms (25 cm×5 cm) and two closed arms (25 cm×5 cm×5 cm) that extend from a central platform (5 cm×5 cm) and are elevated 40 cm from the floor. The apparatus is constructed of clear acrylic over which black duct tape is applied to the walls and floor, which prevents slippage on the open arms. Mice will be placed individually in the center square facing an open arm and allowed to explore the maze for 5 min. An arm entry is counted only when all four paws are inside the arm. Measures scored include (1) time spent in open arms, (2) time spent in closed arms, (3) number of open arm entries, (4) number of closed arm entries, and (5) number of rears in closed arms. Percent of time spent in open arms and total number of entries (to assess locomotor activity) are calculated from the raw data. Time spent in the closed arms is considered an anxiety response.

### Aggression.

Resident-intruder aggression. Aggression will be assessed using a resident-intruder paradigm. Males will be housed individually for one week before aggression is assessed. Aggression will be assessed over three sessions, spaced 2-3 days apart. Resident males will be exposed to different intruder males in each session. An intruder male will be placed in the subject male's home cage for 5 min. Measures will include (1) attack latency, (2) attack duration (total), and (3) number of aggressive bouts. In addition, defensive behaviors, such as flight and defensive supine postures, will be scored if observed. Although only male mice typically display aggression in this type of test, both males and females will be tested (10 of each genotype and treatment group), because normal levels of aggression may be enhanced by genetic manipulation. Female intruder mice will be used instead of males for tests with resident females to eliminate the confounding effects of sexual behaviors.

### Seizure Susceptibility.

Generalized seizures. Susceptibility to generalized, clonic-tonic seizures will be assessed using the convulsant agents, flurothyl and pentylenetetrazole (PTZ), as described in the literature. For flurothyl, mice will be placed in an airtight Plexiglas chamber, and the volatile convulsant 2,2,2-trifluroethylether (flurothyl) will be infused onto filter paper at a rate of 20 μl/min, from which it will vaporize. For PTZ, mice will be injected i.p. with PTZ (30, 40, or 50 mg/kg), placed in a clear container, and closely observed for 10 min. Latency to first myoclonic jerk and clonic-tonic seizure will be used as measurements of seizure susceptibility. Number of mice progressing to tonic extension and death will be used as measurements of maximal seizure severity.

### Locomotor Activity.

Novel environment. Mice will be tested for hyperactivity or inactivity in response to a novel environment as described (Weinshenker et al., 2002). Ambulations (consecutive beam breaks) will be measured in transparent Plexiglas cages (40×20×20 cm) placed into a rack with seven infrared photobeams spaced 5 cm apart, each end beam 5 cm from the cage wall (San Diego Instruments Inc., La Jolla, Calif.). Mice will be placed in the activity chambers for 2 h, and ambulations will be recorded in 30-min bins.

Circadian activity: Mice will be tested for hyperactivity or inactivity over a full circadian cycle by placing them in the activity chambers described above, which are equipped with a food hopper and water bottle. After a 24-h “habituation” phase for the mice to acclimate to their environment, ambulations will be recorded in 30-min bins for one full light cycle and one full dark cycle (24 h).

### Coordinated Movement.

Rotarod. Rotarod tests will be performed as described in the literature. Mice will be placed on the rotarod while stationary. Three consecutive trials will be given each day, except for the first. On the first day the orientation of the mice will be alternated, so that they have to walk backward on the first, third, and fifth trials, and forward on the second, fourth, and sixth trials. On all other days the mice are required to move forward only. Each trial lasts up to 2 min. The average time a mouse remains on the rotarod is calculated for each day. After five days of training at the lowest speed (3.4 rpm), mice will be subjected to an accelerating rotarod. The procedure will be the same, except that the rotarod accelerates over a period of 5 min from 3.4 rpm to 26 rpm (equivalent to a running speed of 8.2 cm/s). Each trial is continued until all mice have fallen off the rod. An average for each mouse over all 12 trials in addition to a daily average will be calculated.

Pole test. The performance of mice in the pole test will be assessed as described in the literature. Mice are placed on top of a wooden pole, which is 50 cm high and 1 cm wide. The base of the pole is placed in the animal's home cage. Time to orient downward and descend the pole is recorded. Mice will receive two days of training (five trials each session). On test day animals will be recorded over five trials and the best of the five performances will be recorded.

Challenging beam traversal. The performance of mice in the challenging beam traversal will be assessed as described in the literature. Mice are trained to walk across a progressively narrowing beam covered with a metal grid. Mice are given two days of training (assisted trials) prior to testing without the mesh grid in which the home cage is positioned closely to the beam as encouragement along the beam until they are able to traverse the entire length of the beam unassisted. Animals will be videotaped throughout five trials and then viewed in slow motion. The total number of steps and time to traverse the beam will be used to rate severity of movement deficit. Errors will include when a limb slips through the grid during a forward movement. An animal can make a maximum of four errors per step. Errors per step, time to traverse, and number of steps will be calculated and averaged across the five trials for each animal.

Gait analysis. Gait activity will be monitored by an automated system (MouseSpecifics, CuraVita). Mice are trained to walk on a clear treadmill over a high-speed camera, and 17 different parameters of gait dynamics (e.g., stride length, brake time, paw angle, stance width, etc) are recorded. Mice will be habituated to the chamber for 5 min/day for five days prior to testing.

### Example II

### Behavioral Experiments on Dmxl1Knock Out Mouse Model

Sixteen wild type (WT) and Dmxl1 heterozygous knock out mice of each sex between 3 and 6 months of age were used for behavioral analysis.

The heterozygous Dmxl1 knockout mouse is impaired in social recognition. The WT and Dmxl1 heterozygous knock out mice were subjected to the same stimulus for the first four consecutive trials. With subsequent trials a decrease in social investigation time is expected if memory exists which is seen in the WT mice. The Dmxl1 heterozygous knock out mice do not demonstrate a decrease in social investigation time. In the last trial, mice were exposed to a novel stimulus and an increase in investigation time back to baseline is expected, which was seen in WT mice.

The heterozygous Dmxl1 knockout mouse does show any difference in circadian cycle in comparison to the WT mouse. The activity of WT and Dmxl1 heterozygous knock out mice was tested over a full circadian cycle. The mice were monitored over a 12 hour dark cycle followed by a 12 hour light cycle. Mice being nocturnal are expected to be more active during dark cycle compared to light cycle. No difference was seen in the activities between WT and Dmxl1 heterozygous knock out mice.

### Example III

### Further Defining the Phenotype Associated with Mutations in the DMXL1 Gene

Extensive clinical evaluation was performed on two DMXL1 patients and their parents. An IRB consent and extensive clinical history was documented on the clinical evaluation form developed specifically for this project. Patient 1 is a 3 year old male carrying a p.Q1926R de novo mutation and Patient 2 is a 6 year old female carrying a p.G2733D de novo mutation.

Patient 1 had hypotonia as an infant, feeding difficulties that did not require a feeding tube, right strabismus, and left cryptorchidism. He had acquired microcephaly (OFC at birth 10^{th} centile; current OFC <<5^{th} centile), normal head MRI, no seizures, and development of lower extremity spasticity. He walked at 18-20 months and is currently nonverbal. He has balance/coordination issues and hyperactivity. He was found to have a Q1926R mutation in DMXL1.

Patient 2 is a 6-9/12 year old female with infantile hypotonia, failure to thrive that necessitated a gastrostomy tube, strabismus, and hypoplastic labia majora. She also has acquired microcephaly (OFC at birth 25^{th}-50^{th} centile; current OFC 2^{rd} centile), normal head MRI, no seizures, and development of lower extremity spasticity. She does not have hyperphagia but weight is at 90^{th} centile. She walked at 3 years and was able to say up to 40 words at 3 years; however, she has since lost all ability to speak. Testing for Rett syndrome and PWS/Angelman syndrome was negative, but DMXL1 nucleic acid sequencing identified a G2733D mutation. She also has balance/coordination issues and hyperactivity.

Both patients have previously received extensive clinical workup including Cytogenetic 44K Oligoarray, Prader Willi and Angelman syndrome analysis, and MeCP2 gene sequencing. They both share some common physical characteristics like hypertelorism, long triangular shape of the face, and arched eyebrows. They also share some clinical features like infantile hypotonia, strabismus, childhood spasticity, microcephaly, hyperactivity and some shared features with PWS (Table 4).

The foregoing is illustrative of the present invention, and is not to be construed as limiting thereof. The invention is defined by the following claims, with equivalents of the claims to be included therein.

All publications, patent applications, patents, patent publications, all sequences identified by GenBank® database and/or SNP accession numbers, and other references cited herein are incorporated by reference in their entireties for the sequences and/or teachings relevant to the sentence and/or paragraph and/or claim in which the reference is presented.

### REFERENCES

1 Schluth, C., Cossee, M., Girard-Lemaire, F., Carelle, N., Dollfus, H., Jeandidier, E. and Flori, E. (2007) Phenotype in X chromosome rearrangements: pitfalls of X inactivation study. Pathol Biol (Paris). 55, 29-36
2 Martin, C. L. and Ledbetter, D. H. (2007) Autism and cytogenetic abnormalities: solving autism one chromosome at a time. Curr Psychiatry Rep. 9, 141-147
3 Raymond, F. L. (2006) X linked mental retardation: a clinical guide. J Med Genet. 43, 193-200
4 Raymond, F. L. and Tarpey, P. (2006) The genetics of mental retardation. Hum Mol Genet. 15 Spec No 2, R110-116
5 Ropers, H. H. (2006) X-linked mental retardation: many genes for a complex disorder. Curr Opin Genet Dev. 16, 260-269
6 Ropers, H. H. and Hamel, B. C. (2005) X-linked mental retardation. Nat Rev Genet. 6, 46-57
7 Engels, H., Brockschmidt, A., Hoischen, A., Landwehr, C., Bosse, K., Walldorf, C., Toedt, G., Radlwimmer, B., Propping, P., Lichter, P. and Weber, R. G. (2007) DNA microarray analysis identifies candidate regions and genes in unexplained mental retardation. Neurology. 68, 743-750
8 Stevenson, R. E. (2005) Advances in X-linked mental retardation. Curr Opin Pediatr. 17, 720-724
9 Dutta, S., Nandagopal, K., Gangopadhyay, P. K. and Mukhopadhyay, K. (2005) Molecular aspects of Down syndrome. Indian Pediatr. 42, 339-344
10 Obe, G. and Ristow, H. (1979) Mutagenic, cancerogenic and teratogenic effects of alcohol. Mutat Res. 65, 229-259
11 Tassone, F., Hagerman, R. J., Loesch, D. Z., Lachiewicz, A., Taylor, A. K. and Hagerman, P. J. (2000) Fragile X males with unmethylated, full mutation trinucleotide repeat expansions have elevated levels of FMR1 messenger RNA. Am J Med Genet. 94, 232-236
12 Fischer, K. M. (1996) Genes for Prader Willi syndrome/Angelman syndrome and fragile X syndrome are homologous, with genetic imprinting and unstable trinucleotide repeats causing mental retardation, autism and aggression. Med Hypotheses. 47, 289-298
13 Lalande, M. and Calciano, M. A. (2007) Molecular epigenetics of Angelman syndrome. Cell Mol Life Sci. 64, 947-960
14 Schmid, A., Moser, H., Walti, U., Herschkowitz, N., Colombo, J. P., Wiesmann, U. and Rossi, E. (1974) [Mental retardation in childhood. Clinical, biochemical and epidemiological studies on 286 institutionalized children]. Schweiz Med Wochenschr. 104, 915-923
15 Rife, M., Mallolas, J., Castellvi-Bel, S., Badenas, C., Jimenez, D. and Mila, M. (2000) Molecular study of the PAK3 and GDI1 genes in nonsyndromic X-linked mental retardation Spanish patients. Am J Med Genet. 94, 389-391
16 Villard, L., Lossi, A. M., Cardoso, C., Proud, V., Chiaroni, P., Colleaux, L., Schwartz, C. and Fontes, M. (1997) Determination of the genomic structure of the XNP/ATRX gene encoding a potential zinc finger helicase. Genomics. 43, 149-155
17 Malinowski, M. J. and Blatt, R. J. (1997) Commercialization of genetic testing services: the FDA, market forces, and biological tarot cards. Tulane Law Rev. 71, 1211-1312
18 Raymond, F. L. (2003) Genetic services for people with intellectual disability and their families. J Intellect Disabil Res. 47, 509-514
19 Camprubi-Sanchez, C., Gabau-Vila, E., Artigas-Pallares, J., Coll-Sandiumenge, M. D. and Guitart-Feliubadalo, M. (2006) [From the clinical to the genetic diagnosis of Prader-Willi and Angelman syndromes.]. Rev Neurol. 42 Suppl 1, S61-67
20 Gunay-Aygun, M., Cassidy, S. B. and Nicholls, R. D. (1997) Prader-Willi and other syndromes associated with obesity and mental retardation. Behav Genet. 27, 307-324
21 Gunay-Aygun, M., Schwartz, S., Heeger, S., O'Riordan, M. A. and Cassidy, S. B. (2001) The changing purpose of Prader-Willi syndrome clinical diagnostic criteria and proposed revised criteria. Pediatrics. 108, E92
22 Driscoll, D. J., Waters, M. F., Williams, C. A., Zori, R. T., Glenn, C. C., Avidano, K. M. and Nicholls, R. D. (1992) A DNA methylation imprint, determined by the sex of the parent, distinguishes the Angelman and Prader-Willi syndromes. Genomics. 13, 917-924
23 Nicholls, R. D., Knoll, J. H., Butler, M. G., Karam, S. and Lalande, M. (1989) Genetic imprinting suggested by maternal heterodisomy in nondeletion Prader-Willi syndrome. Nature. 342, 281-285
24 Varela, M. C., Simoes-Sato, A. Y., Kim, C. A., Bertola, D. R., De Castro, C. I. and Koiffmann, C. P. (2006) A new case of interstitial 6q16.2 deletion in a patient with Prader-Willi-like phenotype and investigation of SIM1 gene deletion in 87 patients with syndromic obesity. Eur J Med Genet. 49, 298-305
25 Faivre, L., Cormier-Daire, V., Lapierre, J. M., Colleaux, L., Jacquemont, S., Genevieve, D., Saunier, P., Munnich, A., Turleau, C., Romana, S., Prieur, M., De Blois, M. C. and Vekemans, M. (2002) Deletion of the SIM1 gene (6q16.2) in a patient with a Prader-Willi-like phenotype. J Med Genet. 39, 594-596
26 Smith, T. F., Gaitatzes, C., Saxena, K. and Neer, E. J. (1999) The WD repeat: a common architecture for diverse functions. Trends Biochem Sci. 24, 181-185
27 Garcia-Higuera, I., Gaitatzes, C., Smith, T. F. and Neer, E. J. (1998) Folding a WD repeat propeller. Role of highly conserved aspartic acid residues in the G protein beta subunit and Sec13. J Biol Chem. 273, 9041-9049
28 Gettemans, J., Meerschaert, K., Vandekerckhove, J. and De Corte, V. (2003) A kelch beta propeller featuring as a G beta structural mimic: reinventing the wheel? Sci STKE. 2003, PE27
29 Guerrini, R. and Carrozzo, R. (2002) Epileptogenic brain malformations: clinical presentation, malformative patterns and indications for genetic testing. Seizure. 11 Suppl A, 532-543; quiz 544-537
30 Morris, S. M., Albrecht, U., Reiner, O., Eichele, G. and Yu-Lee, L. Y. (1998) The lissencephaly gene product Lisl, a protein involved in neuronal migration, interacts with a nuclear movement protein, NudC. Curr Biol. 8, 603-606
31 Ridley, A. J., Colley, J., Wynford-Thomas, D. and Jones, C. J. (2005) Characterisation of novel mutations in Cockayne syndrome type A and xeroderma pigmentosum group C subjects. J Hum Genet. 50, 151-154
32 Zhou, H. X. and Wang, G. (2001) Predicted structures of two proteins involved in human diseases. Cell Biochem Biophys. 35, 35-47
33 Handschug, K., Sperling, S., Yoon, S. J., Hennig, S., Clark, A. J. and Huebner, A. (2001) Triple A syndrome is caused by mutations in AAAS, a new WD-repeat protein gene. Human Molecular Genetics. 10, 283-290
34 Bassi, M. T., Ramesar, R. S., Caciotti, B., Winship, I. M., De Grandi, A., Riboni, M., Townes, P. L., Beighton, P., Ballabio, A. and Borsani, G. (1999) X-linked late-onset sensorineural deafness caused by a deletion involving OA1 and a novel gene containing WD-40 repeats. American Journal of Human Genetics. 64, 1604-1616
35 Gratenstein, K., Heggestad, A. D., Fortun, J., Notterpek, L., Pestov, D. G. and Fletcher, B. S. (2005) The WD-repeat protein GRWD1: potential roles in myeloid differentiation and ribosome biogenesis. Genomics. 85, 762-773
36 Komachi, K., Redd, M. J. and Johnson, A. D. (1994) The WD repeats of Tup1 interact with the homeo domain protein alpha 2. Genes Dev. 8, 2857-2867
37 Yoon, Y. M., Baek, K. H., Jeong, S. J., Shin, H. J., Ha, G. H., Jeon, A. H., Hwang, S. G., Chun, J. S. and Lee, C. W. (2004) WD repeat-containing mitotic checkpoint proteins act as transcriptional repressors during interphase. FEBS Lett. 575, 23-29
38 Li, D. and Roberts, R. (2001) WD-repeat proteins: structure characteristics, biological function, and their involvement in human diseases. Cell Mol Life Sci. 58, 2085-2097
39 Chernova, O. B., Hunyadi, A., Malaj, E., Pan, H., Crooks, C., Roe, B. and Cowell, J. K. (2001) A novel member of the WD-repeat gene family, WDR11, maps to the 10q26 region and is disrupted by a chromosome translocation in human glioblastoma cells. Oncogene. 20, 5378-5392
40 Fritzius, T., Frey, A. D., Schweneker, M., Mayer, D. and Moelling, K. (2007) WD-repeat-propeller-FYVE protein, ProF, binds VAMP2 and protein kinase Czeta. FEBS J. 274, 1552-1566
41 Gunawardane, R. N., Martin, O. C. and Zheng, Y. (2003) Characterization of a new gammaTuRC subunit with WD repeats. Molecular Biology of the Cell. 14, 1017-1026
42 Zaphiropoulos, P. G. and Toftgard, R. (1996) cDNA cloning of a novel WD repeat protein mapping to the 9q22.3 chromosomal region. DNA Cell Biol. 15, 1049-1056
43 Kapranov, P., Willingham, A. T. and Gingeras, T. R. (2007) Genome-wide transcription and the implications for genomic organization. Nat Rev Genet. 8, 413-423
44 Willingham, A. T., Dike, S., Cheng, J., Manak, J. R., Bell, I., Cheung, E., Drenkow, J., Dumais, E., Duttagupta, R., Ganesh, M., Ghosh, S., Helt, G., Nix, D., Piccolboni, A., Sementchenko, V., Tammana, H., Kapranov, P. and Gingeras, T. R. (2006) Transcriptional landscape of the human and fly genomes: nonlinear and multifunctional modular model of transcriptomes. Cold Spring Harb Symp Quant Biol. 71, 101-110
45 Cottrell, S., Bicknell, D., Kaklamanis, L. and Bodmer, W. F. (1992) Molecular analysis of APC mutations in familial adenomatous polyposis and sporadic colon carcinomas. Lancet. 340, 626-630
46 Kinzler, K. W., Nilbert, M. C., Su, L. K., Vogelstein, B., Bryan, T. M., Levy, D. B., Smith, K. J., Preisinger, A. C., Hedge, P., McKechnie, D. and et al. (1991) Identification of FAP locus genes from chromosome 5q21. Science. 253, 661-665
47 Miki, Y., Nishisho, I., Miyoshi, Y., Horii, A., Ando, H., Nakajima, T., Utsunomiya, J. and Nakamura, Y. (1991) Frequent loss of heterozygosity at the MCC locus on chromosome 5q21-22 in sporadic colorectal carcinomas. Jpn J Cancer Res. 82, 1003-1007
48 Kraemer, C., Enklaar, T., Zabel, B. and Schmidt, E. R. (2000) Mapping and structure of DMXL1, a human homologue of the DmX gene from Drosophila melanogaster coding for a WD repeat protein. Genomics. 64, 97-101
49 Bennett, R. L., Karayiorgou, M., Sobin, C. A., Norwood, T. H. and Kay, M. A. (1997) Identification of an interstitial deletion in an adult female with schizophrenia, mental retardation, and dysmorphic features: further support for a putative schizophrenia-susceptibility locus at 5q21-23.1. Am J Hum Genet. 61, 1450-1454
50 Raedle, J., Friedl, W., Engels, H., Koenig, R., Trojan, J. and Zeuzem, S. (2001) A de novo deletion of chromosome 5q causing familial adenomatous polyposis, dysmorphic features, and mild mental retardation. Am J Gastroenterol. 96, 3016-3020
51 Garcia-Minaur, S., Ramsay, J., Grace, E., Minns, R. A., Myles, L. M. and FitzPatrick, D. R. (2005) Interstitial deletion of the long arm of chromosome 5 in a boy with multiple congenital anomalies and mental retardation: Molecular characterization of the deleted region to 5q22.3q23.3. Am J Med Genet A. 132, 402-410
52 Finch, R., Moore, H. G., Lindor, N., Jalal, S. M., Markowitz, A., Suresh, J., Offit, K. and Guillem, J. G. (2005) Familial adenomatous polyposis and mental retardation caused by a de novo chromosomal deletion at 5q15-q22: report of a case. Dis Colon Rectum. 48, 2148-2152
53 van den Boom, J., Wolter, M., Blaschke, B., Knobbe, C. B. and Reifenberger, G. (2006) Identification of novel genes associated with astrocytoma progression using suppression subtractive hybridization and real-time reverse transcription-polymerase chain reaction. Int J Cancer. 119, 2330-2338
54 Nagano, F., Kawabe, H., Nakanishi, H., Shinohara, M., Deguchi-Tawarada, M., Takeuchi, M., Sasaki, T. and Takai, Y. (2002) Rabconnectin-3, a novel protein that binds both GDP/GTP exchange protein and GTPase-activating protein for Rab3 small G protein family. J Biol Chem. 277, 9629-9632
55 Sakisaka, T. and Takai, Y. (2005) Purification and properties of rabconnectin-3. Methods Enzymol. 403, 401-407
56 Kawabe, H., Sakisaka, T., Yasumi, M., Shingai, T., Izumi, G., Nagano, F., Deguchi-Tawarada, M., Takeuchi, M., Nakanishi, H. and Takai, Y. (2003) A novel rabconnectin-3-binding protein that directly binds a GDP/GTP exchange protein for Rab3A small G protein implicated in Ca(2+)-dependent exocytosis of neurotransmitter. Genes Cells. 8, 537-546
57 Castro, L. F., Santos, M. M. and Reis-Henriques, M. A. (2005) The genomic environment around the Aromatase gene: evolutionary insights. BMC Evol Biol. 5, 43
58 Liehr, T., Starke, H., Heller, A., Weise, A., Beensen, V., Senger, G., Kittner, G., Prechtel, M., Claussen, U. and Seidel, J. (2003) Evidence for a new microdeletion syndrome in 15q21. Int J Mol Med. 11, 575-577
59 Muralidhar, B., Marney, A. and Butler, M. G. (1999) Analysis of imprinted genes in subjects with Prader-Willi syndrome and chromosome 15 abnormalities. Genet Med. 1, 141-145
60 Park, W. S., Park, J. Y., Oh, R. R., Yoo, N.J., Lee, S. H., Shin, M. S., Lee, H. K., Han, S., Yoon, S. K., Kim, S. Y., Choi, C., Kim, P. J., Oh, S. T. and Lee, J. Y. (2000) A distinct tumor suppressor gene locus on chromosome 15q21.1 in sporadic form of colorectal cancer. Cancer Res. 60, 70-73
61 Martin, F., Platt, J., Tawn, E. J. and Burn, J. (1990) A de novo interstitial deletion of 15(q21.2q22.1) in a moderately retarded adult male. J Med Genet. 27, 637-639
62 Lalani, S. R., Sahoo, T., Sanders, M. E., Peters, S. U. and Bejjani, B. A. (2006) Coarctation of the aorta and mild to moderate developmental delay in a child with a de novo deletion of chromosome 15(q21.1q22.2). BMC Med Genet. 7, 8
63 Faivre, L., Dollfus, H., Lyonnet, S., Alembik, Y., Megarbane, A., Samples, J., Gorlin, R. J., Alswaid, A., Feingold, J., Le Merrer, M., Munnich, A. and Cormier-Daire, V. (2003) Clinical homogeneity and genetic heterogeneity in Weill-Marchesani syndrome. Am J Med Genet A. 123, 204-207
64 Zhang, Z., Charsky, C., Kane, P. M. and Wilkens, S. (2003) Yeast V1-ATPase: affinity purification and structural features by electron microscopy. J Biol Chem. 278, 47299-47306
65 Kane, P. M. (2005) Close-up and genomic views of the yeast vacuolar H+-ATPase. J Bioenerg Biomembr. 37, 399-403
66 Kane, P. M. (2006) The where, when, and how of organelle acidification by the yeast vacuolar H+-ATPase. Microbiol Mol Biol Rev. 70, 177-191
67 Sambade, M. and Kane, P. M. (2004) The yeast vacuolar proton-translocating ATPase contains a subunit homologous to the Manduca sexta and bovine e subunits that is essential for function. J Biol Chem. 279, 17361-17365
68 Brace, E. J., Parkinson, L. P. and Fuller, R. S. (2006) Skp1p regulates Soi3p/Rav1p association with endosomal membranes but is not required for vacuolar ATPase assembly. Eukaryot Cell. 5, 2104-2113
69 Zhang, J. W., Parra, K. J., Liu, J. and Kane, P. M. (1998) Characterization of a temperature-sensitive yeast vacuolar ATPase mutant with defects in actin distribution and bud morphology. J Biol Chem. 273, 18470-18480
70 Smardon, A. M., Tarsio, M. and Kane, P. M. (2002) The RAVE complex is essential for stable assembly of the yeast V-ATPase. J Biol Chem. 277, 13831-13839
71 Seol, J. H., Shevchenko, A., Shevchenko, A. and Deshaies, R. J. (2001) Skp1 forms multiple protein complexes, including RAVE, a regulator of V-ATPase assembly. Nat Cell Biol. 3, 384-391
72 Michigami, T., Kageyama, T., Satomura, K., Shima, M., Yamaoka, K., Nakayama, M. and Ozono, K. (2002) Novel mutations in the a3 subunit of vacuolar H(+)-adenosine triphosphatase in a Japanese patient with infantile malignant osteopetrosis. Bone. 30, 436-439
73 Frattini, A., Orchard, P. J., Sobacchi, C., Giliani, S., Abinun, M., Mattsson, J. P., Keeling, D. J., Andersson, A. K., Wallbrandt, P., Zecca, L., Notarangelo, L. D., Vezzoni, P. and Villa, A. (2000) Defects in TCIRG1 subunit of the vacuolar proton pump are responsible for a subset of human autosomal recessive osteopetrosis. Nature Genetics. 25, 343-346
74 Fasth, A. and Porras, O. (1999) Human malignant osteopetrosis: pathophysiology, management and the role of bone marrow transplantation. Pediatric Transplantation. 3 Suppl 1, 102-107
75 Sobacchi, C., Frattini, A., Orchard, P., Porras, O., Tezcan, I., Andolina, M., Babul-Hirji, R., Baric, I., Canham, N., Chitayat, D., Dupuis-Girod, S., Ellis, I., Etzioni, A., Fasth, A., Fisher, A., Gerritsen, B., Gulino, V., Horwitz, E., Klamroth, V., Lanino, E., Mirolo, M., Musio, A., Matthijs, G., Nonomaya, S., Notarangelo, L. D., Ochs, H. D., Superti Furga, A., Valiaho, J., van Hove, J. L., Vihinen, M., Vujic, D., Vezzoni, P. and Villa, A. (2001) The mutational spectrum of human malignant autosomal recessive osteopetrosis. Human Molecular Genetics. 10, 1767-1773
76 Sly, W. S., Hewett-Emmett, D., Whyte, M. P., Yu, Y. S. and Tashian, R. E. (1983) Carbonic anhydrase II deficiency identified as the primary defect in the autosomal recessive syndrome of osteopetrosis with renal tubular acidosis and cerebral calcification. Proceedings of the National Academy of Sciences (United States of America). 80, 2752-2756
77 Stover, E. H., Borthwick, K. J., Bavalia, C., Eady, N., Fritz, D. M., Rungroj, N., Giersch, A. B., Morton, C. C., Axon, P. R., Akil, I., Al-Sabban, E. A., Baguley, D. M., Bianca, S., Bakkaloglu, A., Bircan, Z., Chauveau, D., Clermont, M. J., Guala, A., Hulton, S. A., Kroes, H., Li Volti, G., Mir, S., Mocan, H., Nayir, A., Ozen, S., Rodriguez Soriano, J., Sanjad, S. A., Tasic, V., Taylor, C. M., Topaloglu, R., Smith, A. N. and Karet, F. E. (2002) Novel ATP6V1B1 and ATP6V0A4 mutations in autosomal recessive distal renal tubular acidosis with new evidence for hearing loss. Journal of Medical Genetics. 39, 796-803
78 Karet, F. E., Finberg, K. E., Nayir, A., Bakkaloglu, A., Ozen, S., Hulton, S. A., Sanjad, S. A., Al-Sabban, E. A., Medina, J. F. and Lifton, R. P. (1999) Localization of a gene for autosomal recessive distal renal tubular acidosis with normal hearing (rdRTA2) to 7q33-34. American Journal of Human Genetics. 65, 1656-1665
79 Karet, F. E., Finberg, K. E., Nelson, R. D., Nayir, A., Mocan, H., Sanjad, S. A., Rodriguez-Soriano, J., Santos, F., Cremers, C. W., Di Pietro, A., Hoffbrand, B. I., Winiarski, J., Bakkaloglu, A., Ozen, S., Dusunsel, R., Goodyer, P., Hulton, S. A., Wu, D. K., Skvorak, A. B., Morton, C. C., Cunningham, M. J., Jha, V. and Lifton, R. P. (1999) Mutations in the gene encoding B1 subunit of H+-ATPase cause renal tubular acidosis with sensorineural deafness. Nature Genetics. 21, 84-90
80 Steinmetz, L. M., Scharfe, C., Deutschbauer, A. M., Mokranjac, D., Herman, Z. S., Jones, T., Chu, A. M., Giaever, G., Prokisch, H., Oefner, P. J. and Davis, R. W. (2002) Systematic screen for human disease genes in yeast. Nature Genetics. 31, 400-404
81 Sipos, G., Brickner, J. H., Brace, E. J., Chen, L., Rambourg, A., Kepes, F. and Fuller, R. S. (2004) Soi3p/Rav1p functions at the early endosome to regulate endocytic trafficking to the vacuole and localization of trans-Golgi network transmembrane proteins. Molecular Biology of the Cell. 15, 3196-3209
82 Vida, T. A. and Emr, S. D. (1995) A new vital stain for visualizing vacuolar membrane dynamics and endocytosis in yeast. The Journal of Cell Biology. 128, 779-792
83 Spasic, M. V., Kiss, J., Herrmann, T., Kessler, R., Stolte, J., Galy, B., Rathkolb, B., Wolf, E., Stremmel, W., Hentze, M. W. and Muckenthaler, M. U. (2007) Physiologic systemic iron metabolism in mice deficient for duodenal Hfe. Blood. 109, 4511-4517
84 Tsiouris, J. A. and Brown, W. T. (2004) Neuropsychiatric symptoms of fragile X syndrome: pathophysiology and pharmacotherapy. CNS Drugs. 18, 687-703
85 Lundblad, M., Picconi, B., Lindgren, H. and Cenci, M. A. (2004) A model of L-DOPA-induced dyskinesia in 6-hydroxydopamine lesioned mice: relation to motor and cellular parameters of nigrostriatal function. Neurobiol Dis. 16, 110-123
86 Swoap, S. J., Weinshenker, D., Palmiter, R. D. and Garber, G. (2004) Dbh(−/−) mice are hypotensive, have altered circadian rhythms, and have abnormal responses to dieting and stress. Am J Physiol Regul Integr Comp Physiol. 286, R108-113
87 Szot, P., Weinshenker, D., Rho, J. M., Storey, T. W. and Schwartzkroin, P. A. (2001) Norepinephrine is required for the anticonvulsant effect of the ketogenic diet. Brain Res Dev Brain Res. 129, 211-214
88 Weinshenker, D. and Szot, P. (2002) The role of catecholamines in seizure susceptibility: new results using genetically engineered mice. Pharmacol Ther. 94, 213-233
89 Weinshenker, D., Szot, P., Miller, N. S., Rust, N.C., Hohmann, J. G., Pyati, U., White, S. S. and Palmiter, R. D. (2001) Genetic comparison of seizure control by norepinephrine and neuropeptide Y. J Neurosci. 21, 7764-7769
90 Weinshenker, D., Wei, A., Salkoff, L. and Thomas, J. H. (1999) Block of an ether-a-go-go-like K(+) channel by imipramine rescues egl-2 excitation defects in Caenorhabditis elegans. J Neurosci. 19, 9831-9840
91 Weinshenker, D., White, S. S., Javors, M. A., Palmiter, R. D. and Szot, P. (2002) Regulation of norepinephrine transporter abundance by catecholamines and desipramine in vivo. Brain Res. 946, 239-246
92 Weinshenker, D., Garriga, G. and Thomas, J. H. (1995) Genetic and pharmacological analysis of neurotransmitters controlling egg laying in C. elegans. J Neurosci. 15, 6975-6985
93 Weinshenker, D., Miller, N. S., Blizinsky, K., Laughlin, M. L. and Palmiter, R. D. (2002) Mice with chronic norepinephrine deficiency resemble amphetamine-sensitized animals. Proceedings of the National Academy of Sciences (United States of America). 99, 13873-13877
94 Weinshenker, D., Rust, N.C., Miller, N. S. and Palmiter, R. D. (2000) Ethanol-associated behaviors of mice lacking norepinephrine. J Neurosci. 20, 3157-3164
95 Weinshenker, D. and Schroeder, J. P. (2006) There and Back Again: A Tale of Norepinephrine and Drug Addiction. Neuropsychopharmacology. 32, 1433-1451
96 Weinshenker, D., Wilson, M. M., Williams, K. M., Weiss, J. M., Lamb, N. E. and Twigger, S. N. (2005) A new method for identifying informative genetic markers in selectively bred rats. Mamm Genome. 16, 784-791
97 Rambourg, A., Clermont, Y. and Kepes, F. (1993) Modulation of the Golgi apparatus in Saccharomyces cerevisiae sec7 mutants as seen by three-dimensional electron microscopy. The Anatomical Record. 237, 441-452
98 Prescianotto-Baschong, C. and Riezman, H. (1998) Morphology of the yeast endocytic pathway. Molecular Biology of the Cell. 9, 173-189
99 Brickner, J. H. and Fuller, R. S. (1997) SOI1 encodes a novel, conserved protein that promotes TGN-endosomal cycling of Kex2p and other membrane proteins by modulating the function of two TGN localization signals. The Journal of Cell Biology. 139, 23-36

TABLE 1

| | Prader-Willi | Deletion 5q21-q23 | Deletion 15q21 |
|---|---|---|---|
| | Syndrome | (including | (including |
| Feature | (PWS) | DMXL1) | DMXL2) |
| Mental retardation | + | + | + |
| Hypotonia | + | + | + |
| Failure to thrive | + | + | − |
| Later onset obesity | + | + | + |
| Prominent | + | + | + |
| forehead | | | |
| Low-set, dysplastic | − | + | + |
| ears | | | |
| Palatal anomalies | − | + | − |
| Down-turned | + | + | − |
| corners of the mouth | | | |
| Receding chin/ | − | + | + |
| micrognathia | | | |
| Small hands/feet | + | − | + |
| Tapered fingers | + | + | + |
| Camptodactyly | − | + | − |
| Genital | + | − | + |
| malformations | | | |

“+” = present; “−” = absent

TABLE 2

| | | | | | | |
|---|---|---|---|---|---|---|
| | Nucleotide | Amino Acid | Ensembl | | Yeast Rav1 | |
| Exon | Change | Change | rs no. | SIFT/PANTER/POLYPHEN | Conservation | De novo |

DMXL1 MISSENSE CHANGES

| | | | | | | |
|---|---|---|---|---|---|---|
| 24 | 5476 C > T | R1826C | Not SNP | Damaging | Yes | Yes |
| 24 | 5947 A > G | M1983V | Not SNP | Probably Damaging | No | Yes |
| 24 | 6166 C > T | R2056C | Not SNP | Damaging | Yes | Yes |
| 11 | 1462 A > G | K488E | Not SNP | Possibly Damaging | Yes | Not Yet Determined |
| 18 | 3716 G > C | C1239S | Not SNP | Possibly Damaging | No | Not Yet Determined |
| 20 | 4765 A > G | M1589V | Not SNP | Possibly Damaging | Yes | Not Yet Determined |
| 23 | 5321 G > A | S1774N | Not SNP | Probably Damaging | Yes (putative | Not Yet Determined |
| | | | | | functional domain) | |
| 24 | 5777 A > G | Q1926R | Not SNP | Benign | No | Not Yet Determined |
| 24 | 6225 C > G | D2075E | Not SNP | Probably Damaging | Yes | Not Yet Determined |
| 27 | 6839 G > T | S2280I | Not SNP | Damaging | Yes | Not Yet Determined |
| 36 | 8198 G > A | G2733D | Not SNP | Possibly Damaging | Yes | Not Yet Determined |

DMXL2 MISSENSE CHANGES

| | | | | | | |
|---|---|---|---|---|---|---|
| 18 | 4466 G > T | R1489I | Not SNP | Possibly Damaging | Yes (putative | Not Yet Determined |
| | | | | | functional domain) | |
| 24 | 6032 A > T | D2011V | Not SNP | Possibly Damaging | No | Not Yet Determined |

TABLE 3

| Locus | Chromosome | Start | End | Size |
|---|---|---|---|---|
| 1p telomere | 1 | 849,476 | 4,915,415 | 4,065,939 |
| 2q telomere | 2 | 237,960,273 | 242,533,697 | 4,573,424 |
| DMXL1 | 5 | 118,429,983 | 118,617,721 | 187,738 |
| 6q16.2 | 6 | 100,943,472 | 101,018,272 | 74,800 |
| 12q telomere | 12 | 126,419,303 | 132,385,722 | 5,966,419 |
| PWS/AS | 15 | 20,249,887 | 26,232,997 | 5,983,110 |
| DMXL2 | 15 | 49,522,233 | 49,707,259 | 185,026 |

TABLE 4

| Feature | Patient 1 | Patient 2 |
|---|---|---|
| Hypotonia in infancy | + | + |
| Feeding difficulties in infancy | + | − |
| Failure to Thrive | − | +, necessitating |
| | | G tube when older |
| Weight >90th centile in childhood | − | + |
| Hyperphagia | − | − |
| Strabismus | + | + |
| Hypoplastic genitalia | +, L | +, hypoplastic |
| | cryptorchidism | labia majora |
| Spasticity in childhood | + | + |
| Unsteady gait with balance/ | + | + |
| coordination problems | | |
| Acquired microcephaly | + | + |
| Normal head MRI | + | + |
| Seizures | − | − |
| Nonverbal | + | +, had 40 words |
| | | but lost all words |
| | | at 3 years |
| Hyperactivity | + | + |

TABLE 5

| | | | Endocytic Delivery |
|---|---|---|---|
| Background | Plasmid | ZnCl₂ | to Vacuole |
| WT | M998I | Not tested | Affected |
| WT | Q1926R | Not tested | Affected |
| WT | ΔAAL | Not tested | Affected |
| WT | RAV1 | Not tested | Normal |
| WT | Vector | Not tested | Normal |
| Δrav1 | M998I | +/− | Affected |
| Δrav1 | Q1926R | +/− | Affected |
| Δrav1 | ΔAAL | − | Affected |
| Δrav1 | RAV1 | + | Normal |
| Δrav1 | Vector | − | Affected |

+: growth (+, +/−, −)

That which is claimed is:

1. A method of identifying a human subject as having an increased likelihood of having DMXL-associated mental retardation, comprising detecting, in a nucleic acid sample from the subject, a mutation in a nucleotide sequence encoding DMXL1 and/or a mutation in a nucleotide sequence encoding DMXL2.

2. A method of identifying a human subject as having DMXL-associated mental retardation, comprising detecting, in a nucleic acid sample from the subject, a mutation in a nucleotide sequence encoding DMXL1 and/or a mutation in a nucleotide sequence encoding DMXL2.

3. The method of claim 1, wherein the mutation is a missense mutation.

4. The method of claim 2, wherein the mutation is a missense mutation.

5. The method of claim 1, wherein the mutation is detected by a method selected from the group consisting of sequencing, electrophoretic mobility, nucleic acid hybridization, fluorescence in situ hybridization, polymerase chain reaction, reverse transcription-polymerase chain reaction, denaturing high-performance liquid chromatography, CGH array and any combination thereof.

6. A method of identifying a human subject as having an increased likelihood of having DMXL-associated mental retardation, comprising detecting, in a sample from the subject, a mutation in a DMXL1 protein and/or a mutation in a DMXL2 protein.

7. A method of identifying a human subject as having DMXL-associated mental retardation, comprising detecting, in a sample from the subject, a mutation in a DMXL1 protein and/or a mutation in a DMXL2 protein.

8. The method of claim 6, wherein the mutation is a missense mutation.

9. The method of claim 7, wherein the mutation is a missense mutation.

10. The method of claim 6, wherein the mutation is detected by a method selected from the group consisting of sequencing, immunoassay, molecular weight analysis, electrophoresis and any combination thereof.

11. The method of claim 1, wherein the mutation disrupts the function of the DMXL1 protein and/or the DMXL2 protein.

12. The method of claim 6, wherein the mutation disrupts the function of the DMXL1 protein and/or the DMXL2 protein.

13. The method of claim 1, wherein the subject has a DMXL-associated mental retardation phenotype.

14. The method of claim 13, wherein the subject has tested negative for Prader Willi syndrome, Rett syndrome and/or Angelman syndrome.

15. The method of claim 6, wherein the subject has a DMXL-associated mental retardation phenotype.

16. The method of claim 15, wherein the subject has tested negative for Prader Willi syndrome, Rett syndrome and/or Angelman syndrome.

17. A method of amplifying a segment of a nucleotide sequence encoding DMXL1, comprising:
a) choosing a first oligonucleotide primer from a nucleic acid sequence comprising the nucleotide sequence of SEQ ID NO:1, the nucleotide sequence of SEQ ID NO:3 or the nucleotide sequence of SEQ ID NO:5;
b) choosing a second oligonucleotide primer from a nucleic acid sequence comprising the nucleotide sequence of SEQ ID NO:1, the nucleotide sequence of SEQ ID NO:3 or the nucleotide sequence of SEQ ID NO:5 that differs in nucleotide sequence from the first oligonucleotide primer;
c) adding said first oligonucleotide primer and said second oligonucleotide primer to a nucleic acid sample; and
d) amplifying a segment of the nucleotide sequence encoding DMXL1 defined by said first oligonucleotide primer and said second oligonucleotide primer, wherein said nucleic acid sample is from a subject having a DMXL-associated mental retardation phenotype.

18. A method of amplifying a segment of a nucleotide sequence encoding DMXL2, comprising:
a) choosing a first oligonucleotide primer from a nucleic acid sequence comprising the nucleotide sequence of SEQ ID NO:2, the nucleotide sequence of SEQ ID NO:4 or the nucleotide sequence of SEQ ID NO:7;
b) choosing a second oligonucleotide primer from a nucleic acid sequence comprising the nucleotide sequence of SEQ ID NO:1, the nucleotide sequence of SEQ ID NO:3 or the nucleotide sequence of SEQ ID NO:5 that differs in nucleotide sequence from the first oligonucleotide primer;
c) adding said first oligonucleotide primer and said second oligonucleotide primer to a nucleic acid sample; and
d) amplifying a segment of the nucleotide sequence encoding DMXL2 defined by said first oligonucleotide primer and said second oligonucleotide primer, wherein said nucleic acid sample is from a subject having a DMXL-associated mental retardation phenotype.
